(19) United States
(12) Patent Application Publication
(10) Pub. No.: US 2024/0285534 A1
(43) Pub. Date: Aug. 29, 2024

(54) CO-AMORPHOUS SOLID FORMS OF FLAVONOIDS

(71) Applicant: ALPARIS, S.A. de C.V., Mexico City (MX)

(72) Inventors: Jorge Guillermo DOMÍNGUEZ CHÁVEZ, Boca del Rio (MX); Karina MONDRAGÓN VÁSQUEZ, Boca del Rio (MX); Juan Pablo SENOSIAIN PELÁEZ, Mexico City (MX)

(21) Appl. No.: 18/516,509

(22) Filed: Nov. 21, 2023

Related U.S. Application Data
(60) Provisional application No. 63/428,243, filed on Nov. 28, 2022.

(51) Int. Cl. A61K 9/16; A61K 31/353; A61P 3/00; C07D 311/30

(52) U.S. Cl. CPC A61K 9/1617; A61K 31/353; A61P 3/00; C07D 311/30

(57) ABSTRACT

The present invention relates to new solid forms (NSF) of a flavonoid and a coformer. The NSF are co-amorphous solids of flavonoids and amino acids, as well as the solvates of said co-amorphous solids. The flavonoid is selected from quercetin, genistein, fisetin, apigenin, naringenin, rutin, diosmin, among others. The coformer is selected from lysine and arginine. The NSF have enhanced stability, solubility and pharmacological properties over parent polyphenols. The new solid forms are suitable to be used in pharmaceutical compositions, food products, nutritional supplements and the like.

### CROSS-REFERENCE TO RELATED APPLICATIONS

This application claims priority to U.S. Provisional Application No. 63/428,243 filed Nov. 28, 2022, which is hereby incorporated by reference in its entirety.

### TECHNICAL FIELD OF INVENTION

The present disclosure relates to new co-amorphous solid forms of a polyphenol, specifically a flavonoid, and a coformer, with improved stability, solubility and pharmacological properties over parent polyphenols. The coformer is selected from amino acids or nicotinic acid derivatives. The flavonoid compound is selected from flavonols, flavanones, isoflavones, flavones, flavan-3-ols and anthocyanins, e.g., quercetin, fisetin, rutin, naringenin, genistein, apigenin, diosmin and diosmetin. The coformer is an amino acid selected from arginine and lysine.

### BACKGROUND OF THE INVENTION

Despite the exponential progress in the development of active ingredients, there is a remaining need of having natural therapeutic alternatives with antioxidant, anti-inflammatory, analgesic, antitumor and anticancer effects, among others.

Polyphenols are a group of phytochemical compounds with potential health-promoting effects. Polyphenols are classified into flavonoids, stilbenes and phenolic acids. In the case of flavonoids, the literature generally classifies them into flavonols, flavanones, isoflavones, flavones, flavan-3-ols (flavanols) and anthocyanins.

Flavonoids represent a group of naturally occurring phenolic compounds that are characterized by a structure consisting of two benzene rings (A and B) linked through a pyran ring. Some flavonoids of interest due to their medicinal properties are listed below:

Quercetin (QCT) is one of the most abundant flavonoids, it is a bioflavonoid usually found in onions, apples and several fruit juices. Different studies report an antioxidant effect, an effect on cardiovascular diseases, anti-inflammatory and antitumor activity, etc.

Fisetin (FST) (3,7,3′,4′-tetrahydroxyflavone) is a flavonol. It can be found in strawberries, apples, persimmons, onions and cucumbers. It was first described in 1891.

Rutin (RTN) is also known as rutoside, rutinoside and quercetin-3-rutinoside. It has been found in species of the genera Rheum and Asparagus, and also in some fruits, especially citrus. It is sometimes referred to as vitamin P, but it is not strictly a vitamin. The therapeutic use of rutin is relatively limited due to its extremely low solubility in water (0.125 g/L).

Naringenin (NRG) belongs to the flavanone class. It is extracted from the peel of some citrus fruits and is mainly responsible of their bitter taste. It is also located in the pulp of fruits, in leaves, flowers and seeds. It is found in food supplements, promoted as an enhancer of other supplements, which increases physical capacity and acts as a “fat burner” for weight loss, among other effects.

Genistein (GTN) is an aglycone of the plant isoflavone family. It is found naturally in soybeans. Isoflavones have chemical structures similar to human estrogens. Some publications mention that genistein could be useful in bone and brain health, to prevent heart disease, to reduce the occurrence of breast and prostate cancer, and to prevent hypertension. One of the mechanisms of action is the reduction of oxidative stress, the promotion of growth factor signaling and immune suppression in endothelial, glial and neuronal cells.

Apigenin (APG) (4′,5,7-trihydroxyflavone) belongs to the flavone class. It is a yellow crystalline solid that has been used to dye wool. Apigenin is found in fruits and vegetables such as parsley, celery, chamomile, among others.

Diosmin (DIS) (diosmetin 7-O-rutinoside) is a flavone glycoside of diosmetin, is manufactured from citrus fruit peels as a phlebotonic non-prescription dietary supplement used to aid treatment of hemorrhoids or chronic venous diseases, mainly of the legs. Recently, extensive study has indicated that diosmin possesses diverse pharmacological activities, including anti-inflammation, anti-oxidation, anti-diabetes, anti-cancer, anti-microorganism, liver protection, neuro-protection, cardiovascular protection, renoprotection, and retinal protection activities. Due to its low water solubility, diosmin is dramatically limited in clinical application. Diosmin is a greyish-yellow to light yellow hygroscopic powder, odorless, practically insoluble in water. Due to its low water solubility, diosmin is dramatically limited in clinical application.

Diosmetin (DSMT) (3′,5,7-trihydroxy-4′-methoxyflavone) is an O-methylated flavone, the aglycone part of the flavonoid glycosides diosmin occurs naturally in citrus fruit. Although it is found in herbal medicines and plays an important role in the treatment of various ailments, only limited scientific researches have been conducted. Diosmetin, a citrus flavonoid, has a variety of therapeutic properties such as antibacterial, anti-inflammatory and antioxidant effects. However, it has low hydrophilicity and poor solubility in water resulting in reduced gastrointestinal tract absorption and limited applications in the food and medicine industries.

Flavonoids have several therapeutic applications, but most of them commonly exhibit limited oral bioavailability, probably due to their poor solubility, low permeability and low stability, which severely reduces their efficacy as therapeutic agents. For example, due to the double bond between positions 2 and 3 of flavones and flavonols, they are susceptible of forming flat structures, leading to a narrow molecular arrangement and, consequently, it is difficult for the solvent molecule to penetrate their molecular structures. The aforesaid features limit their use in the development of pharmaceutical formulations, food products and nutritional supplements.

Moreover, due to the presence of hydroxyl and ketone groups and unsaturated double bonds, flavonoids are also sensitive to physical environmental stress (e.g., heat, light) and physiological stress (e.g., digestive enzymes, acidic pH), which likely leads to degradation or biotransformation during their processing, storage and systemic circulation. Therefore, their efficacy is seriously limited through oral absorption.

There are different known technologies applied to the processing of flavonoids to improve their solubility, bioavailability and/or absorption, such as the formation of prodrugs, glycosylation, formation of complexes, use of nanotechnology, etc. The formation or preparation of co-amorphous solids is also a technique that allows to increase the solubility, bioavailability and stability of this type of compounds.

The present invention proposes the formation of new amorphous solid forms comprising a flavonoid compound and a coformer, wherein the coformer may be selected from amino acids such as arginine and lysine.

Arginine is involved in many activities of the endocrine glands, such as stimulating immune function by increasing the number of leukocytes. It is also involved in the synthesis of creatine and polyamines, collagen and DNA (deoxyribonucleotide acid) production, and can also lower cholesterol. It can also stimulate the release of growth hormone, somatotropin. It has a vasodilator effect. L-arginine is converted in the body into nitric oxide, which causes blood vessels to open wider to improve blood flow.

Like L-carnitine, L-arginine (L-Arg) has potentiating functions in the use of fatty acids as energy (muscle fuel). L-arginine can lower cholesterol by improving the capacity of the circulatory system, as well as stimulate the release of growth hormone, reduce body fat levels and facilitate the recovery of athletes (resulting from anaerobic exercise) from muscles and convert it into urea that is excreted in the urine. The recommended oral dose of L-arginine is 500 mg to 6,000 mg daily.

Lysine is an α-amino acid that plays several roles in humans, the most important being proteinogenesis, but also in the crosslinking of collagen polypeptides, uptake of essential mineral nutrients, and in the production of carnitine, which is key in fatty acid metabolism. Lysine is also often involved in histone modifications, and thus, impacts in the epigenome. The human body cannot synthesize lysine. It is essential in humans and must therefore be obtained from the diet.

The inventions described in this application are not reported in any prior art document. Some documents related to the present invention are the following:

Pranali Hatwar et al., Journal of Drug Delivery Science and Technology 62: 102350, “Pellets containing quercetin amino acid co-amorphous mixture for the treatment of pain: Formulation, optimization, in-vitro and in-vivo study”, 2021. This document discloses a co-amorphous mixture QCT:ARG (1:2) which was formulated in pellets using extrusion spheronization method.

CN106220599 relates to a quercetin-lysine amorphous substance.

WO2019208574 discloses a composition containing L-arginine and a glycosyl compound whit a formed of a monosaccharide, a disaccharide, or an oligosaccharide including three to five monosaccharides.

WO2021167580 discloses a water-soluble solid dispersion of quercetin with polyvinyl pyrrolidone (PVP) and alkaline agent, which is characterized by high bioavailability for manufacture of injectable and oral medicinal products.

### SUMMARY OF THE INVENTION

The present invention offers new co-amorphous solid forms (NSF) of flavonoids with amino acids, and solvates thereof, which have improved properties of solubility and stability, allowing to take advantage and/or potentiate their therapeutic effects such as their antioxidant, anti-inflammatory, analgesic, antitumoral, anticancer, effects, among others.

The present invention also relates to pharmaceutical compositions, food products and nutritional supplements comprising new co-amorphous solid forms of flavonoids mentioned above.

The invention also relates to a method for improving health or nutrition of an animal or human comprising administering a composition containing the new solid forms.

The new amorphous solid forms comprise a flavonoid and a coformer in a molar ratio of 1:1 to 1:10. In a preferred embodiment, the flavonoid-to-coformer ratio is less than 1:8. In a preferred embodiment, the flavonoid-to-coformer ratio is less than 1:6. In an even more preferred embodiment, the flavonoid to coformer ratio is 1:3 to 1:4.

The method used to prepare the new solid forms of the present invention is selected from rapid evaporation, dry grinding, water grinding and slurry.

### BRIEF DESCRIPTION OF THE DRAWINGS

FIG. 1. X-ray powder diffractograms of quercetin (QCT), arginine (ARG), physical quercetin-arginine (QCT:ARG) mixture, and the new co-amorphous solid form of quercetin:arginine (QCT:ARG) with molar ratio 1:8.

FIG. 2. FT-IR spectra of quercetin (QCT), arginine (ARG), physical mixture of quercetin-arginine (QCT:ARG), and co-amorphous QCT:ARG 1:8 NSF.

FIG. 3a. DSC-TGA thermogram of QCT:ARG 1:8 NSF.

FIG. 3b. DSC-TGA thermogram of QCT:ARG 1:3 NSF.

FIG. 4. X-ray powder diffractograms of NSF QCT:ARG 1:3 to 1:9.

FIG. 5. FT-IR spectra of NSF QCT:ARG 1:3 to 1:9.

FIG. 6. X-ray powder diffractograms of fisetin (FST), arginine (ARG), physical mixture of fisetin-arginine (FST:ARG), and the co-amorphous NSF fisetin:arginine (FST:ARG) 1:8.

FIG. 7. FT-IR spectra of fisetin (FST), arginine (ARG), physical mixture of fisetin-arginine (FST:ARG) 1:8, and co-amorphous FST:ARG NSF with stoichiometry 1:8.

FIG. 8a. DSC-TGA thermogram of FST:ARG 1:8 NSF.

FIG. 8b. DSC-TGA thermogram of FST:ARG 1:3 NSF.

FIG. 9. X-ray powder diffractograms of NSF FST:ARG 1:3 to 1:9.

FIG. 10a. FT-IR spectra of NSF FST:ARG 1:3 to 1:9.

FIG. 10b. NMR 1H spectrum in D₂O of co-amorphous solvate FIS:ARG:ethanol 1:3:1.

FIG. 11. X-ray powder diffractograms of rutin (RTN), Arginine (ARG) and rutin:arginine (RTN:ARG) NSF with stoichiometries 1:1 to 1:4.

FIG. 12. FT-IR spectra of NSF RTN:ARG 1:1 to 1:4.

FIGS. 13a-13d. DSC-TGA thermograms of RTN (FIG. 13a) and the three NSF RTN:ARG 1:2 (FIG. 13b), 1:3 (FIG. 13c) and 1:4 (FIG. 13d).

FIG. 13e. NMR 1H spectrum in D₂O of co-amorphous solvate RTN:ARG:ethanol 1:3:1.

FIG. 14. X-ray powder diffractograms of genistein (GTN), Arginine (ARG) and the NSF of genistein:arginine (GTN:ARG) 1:8, 1:9 and 1:10 obtained by slurry in water.

FIG. 15. X-ray powder diffractograms of GTN:ARG 1:4 NSF obtained by the grinding method at 5, 10 and 30 minutes.

FIG. 16. X-ray powder diffractograms of GTN:ARG 1:4 NSF obtained by the method of wet grinding at 5 and 30 minutes.

FIG. 17. X-ray powder diffractograms of GTN:ARG 1:4 NSF obtained by the methods of wet grinding at 30 minutes, slurry with 1 drop of water, and slurry with 4 drops of water.

FIG. 18. FT-IR spectra of the raw materials genistein (GTN), arginine (ARG) and solids with ratio GTN:ARG 1:4 obtained by rapid evaporation (RE) of water, wet grinding and slurry in water.

FIG. 19. X-ray powder diffractograms of the GTN:ARG 1:4 NSF obtained by rapid evaporation of water/ethanol, and the one obtained by wet grinding.

FIG. 20. FT-IR spectra of GTN:ARG 1:4 solids obtained by dry grinding and rapid evaporation in ethanol/water, as well as GTN:ARG 1:4 semisolids obtained from rapid evaporation in water and slurry in water.

FIG. 21. Thermal analysis of the solid GTN:ARG 1:4 obtained by rapid evaporation of ethanol/water.

FIG. 22. X-ray powder diffractograms of the raw materials Arginine (ARG) and naringenin (NRG), as well as NSF of naringenin:arginine (NRG:ARG) 1:3.

FIG. 23. FT-IR spectra obtained for NRG:Arg 1:3 NSF and the raw materials ARG and NRG.

FIG. 24a. Thermal analysis of naringenin (left) and the new solid co-amorphous form of NRG:ARG 1:3 (right).

FIG. 24b. NMR ¹H spectrum in D₂O of the co-amorphous solvate NRG:ARG:ethanol 1:3:1.

FIG. 25. X-ray powder diffractograms of the raw materials arginine (ARG) and apigenin (APG), and the products obtained from dry slurry, slurry in ether and slurry in THF.

FIG. 26. FT-IR spectra of the raw materials arginine (ARG) and apigenin (APG), and the products obtained from dry slurry, slurry in ether and slurry in THF.

FIG. 27: FT-IR spectra of the raw materials apigenin (APG) and arginine (ARG), and the product obtained from rapid evaporation of water and DMSO.

FIG. 28a. X-ray powder diffractograms of the solid apigenin:arginine (APG:ARG) 1:8 obtained by rapid evaporation and dry grinding, and the corresponding raw materials.

FIG. 28b. X-ray powder diffractograms of the solid apigenin:arginine (APG:ARG) 1:8 obtained by dry grinding and the physical mixture apigenin-arginine (APG:ARG).

FIG. 29: FT-IR spectra of the raw materials apigenin (APG) and arginine (ARG), and the solids APG:ARG 1:8 obtained by rapid evaporation and dry grinding.

FIG. 30: DSC and TGA thermogram of the new solid co-amorphous form APG:ARG 1:8 obtained by rapid evaporation of water.

FIG. 31. X ray powder diffractograms of the raw materials diosmin (DIS) and arginine (ARG) and the products obtained from dry slurry, slurry in THF and slurry in ether.

FIG. 32. FT-IR spectra of raw materials diosmin (DIS) and arginine (ARG) and the products obtained from dry slurry, slurry in THF and slurry in ether.

FIG. 33. X-ray powder diffractograms of the raw materials diosmin (DIS) and arginine (ARG) and the solid obtained by rapid evaporation (RE) of water and DMSO.

FIG. 34. X-ray powder diffractograms of the raw materials lysine (LYS) and rutin (RTN) and the solid with stoichiometry 1:3 obtained by rapid evaporation (RE) of water.

FIG. 35. FT-IR spectra of the raw materials lysine (LYS) and rutin (RTN) and the solid with 1:3 stoichiometry obtained by rapid evaporation (RE) of water.

FIG. 36. X-ray powder diffractograms of the raw materials lysine (LYS) and naringenin (NRG) and the solid with stoichiometry 1:3 obtained by rapid evaporation of water.

FIG. 37. FT-IR spectra of the raw materials lysine (LYS) and naringenin (NRG) and the solid with 1:3 stoichiometry obtained by rapid evaporation of water.

FIG. 38. X-ray powder diffractograms of the raw materials arginine (ARG) and apigenin (APG), and the solid with stoichiometry 1:3 obtained by rapid evaporation (RE) of water and ethanol.

FIG. 39. FT-IR spectra of the raw materials arginine (ARG) and apigenin (APG), and the solid with stoichiometry 1:3 obtained by rapid evaporation (RE) of water and ethanol.

FIG. 40. Effect of consumption of High-Fat Sugar Diet (HFSD) for each Flavonoid NSF on food intake and weight gain.

FIG. 41. Comparison of fat mass gain in mice fed with HFSD-Flavonoid NSF at three months.

FIG. 42. Comparison of lean mass gain in mice fed with HFSD-Flavonoid NSF at three months.

FIG. 43. Quantification of the area under the curve (AUC), glucose in mice with induced obesity, fed with HFSD-Flavonoid NSF.

FIG. 44. Area under the curve (AUC) of glucose in mice with induced obesity, fed with HFSD-Flavonoid NSF.

FIG. 45. Insulin in mice with induced obesity, fed with HFSD-flavonoids NSF.

FIG. 46. Respiratory Exchange Ratio (RER) in mice fed with HFSD-Flavonoid NSF.

FIG. 47. Consumption of VO2 in mice fed with HFSD-Flavonoid NSF.

FIG. 48. Concentration of LDL in mice fed with HFSD-Flavonoid NSF.

FIG. 49. Concentration of liver enzyme AST U/L (aspartate transaminase) in mice fed with HFSD-Flavonoid NSF.

FIG. 50. Concentration of liver enzyme ALT U/L (alanine transaminase) in mice fed with HFSD-Flavonoid NSF.

FIG. 51. Concentration of urea in mice fed with HFSD-Flavonoid NSF.

FIG. 52. Concentration of creatinine in mice fed with HFSD-Flavonoid NSF.

### DETAILED DESCRIPTION OF THE INVENTION

The present invention relates to co-amorphous solid compounds formed by a flavonoid selected from quercetin, fisetin, rutin, naringenin, genistein, apigenin and diosmin, and an amino acid coformer compound selected from arginine and lysine, and solvates of said co-amorphous solid compounds, wherein the molar ratio of flavonoid:amino acid is between 1:1 and 1:10.

The present invention also relates to food products, nutritional supplements and the like comprising the co-amorphous compounds of flavonoids mentioned above.

The present invention also relates to pharmaceutical compositions comprising co-amorphous solids of flavonoids, and pharmaceutically acceptable vehicles and/or excipients; and the use of said compositions in conditions that require drugs with antioxidant, anti-inflammatory, analgesic, antitumor or anticancer effect, among others.

The present invention also relates to co-amorphous solids of flavonoids with better solubility properties than the flavonoid alone, which allows to improve its absorption and bioavailability. The present invention also relates to co-amorphous solids flavonoids with improved stability, allowing their use in pharmaceutical formulations, nutritional supplements, food products and the like.

When referring to the solid forms of the present invention, the terms “solid form”, “solid phase”, “new solid phase”, “new solid form”, “new solid form”, “NSF” or simply “phase” are used interchangeably in the present application.

The amino acid coformer can be found in configuration L, in configuration D, or as a racemic mixture DL.

The method used to prepare the NSF of the present invention is selected from the group of rapid evaporation, wet grinding, dry grinding and slurry.

The solids obtained with different flavonoids in different ratios were characterized by X-ray powder diffraction and FT-IR. The obtained spectra were compared with the respective raw materials and with the physical mixtures of the two components, to determine if any NSF was obtained from the respective flavonoid. The obtained NSF were also characterized by thermal analysis (DSC TGA).

### Example 1. New Co-Amorphous Solid Form of Quercetin:Arginine (QCT:ARG) 1:8

An NSF of Quercetin:Arginine (QCT:ARG) with a ratio of 1:8 was obtained by the method of rapid evaporation of water.

### Physical Analysis of the QCT:ARG 1:8 NSF

The QCT:ARG NSF obtained with 1:8 stoichiometry is a dark orange solid.

### X-Ray Powder Diffraction of the NSF QCT:ARG 1:8

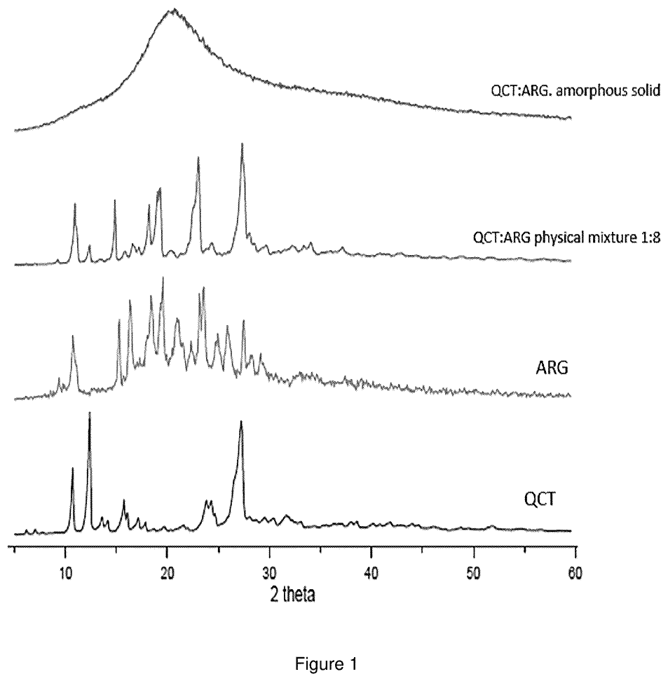

FIG. 1 presents the X-ray powder diffractogram for the raw materials, the physical mixture of quercetin and arginine, and the QCT:ARG 1:8 NSF. The spectra of the raw materials quercetin and arginine exhibit diffraction peaks. The physical mixture shows a pattern of diffraction peaks similar to that of arginine. In contrast, the diffractogram of the solid QCT:ARg 1:8 shows a diffuse halo without diffraction peaks characteristic of an amorphous solid compound. This result together with the color of the solid obtained, allows concluding that it is an amorphous solid form.

### FT-IR Spectroscopy of QCT:ARG 1:8 NSF

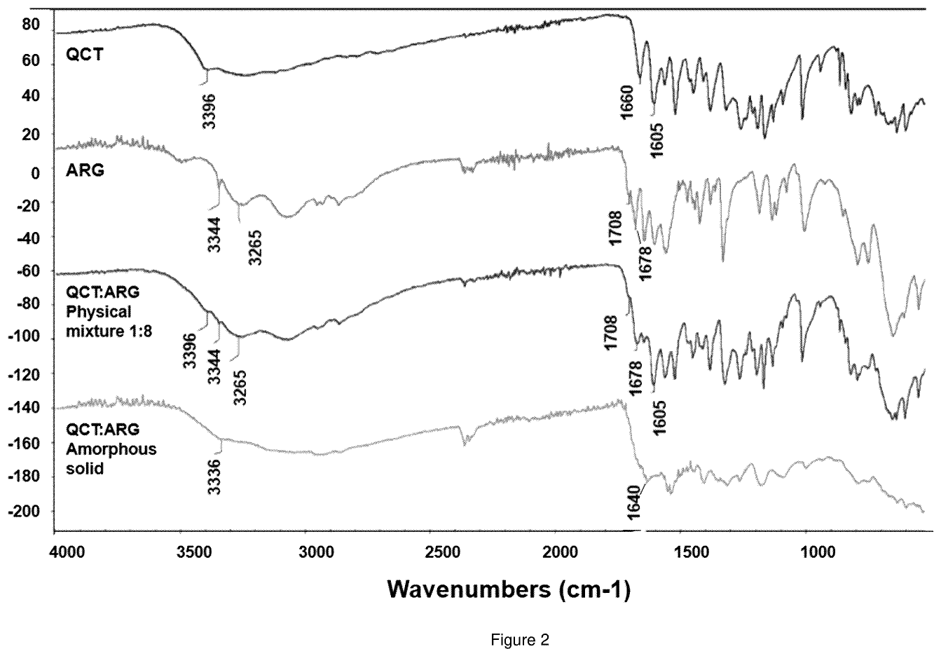

A FT-IR study was conducted to determine the possible intermolecular interactions between quercetin and the coformer. FIG. 2 shows the spectra of quercetin, arginine, the QCT:ARG NSF with stoichiometry 1:8, and the physical mixture of quercetin and arginine, to analyze changes in vibrational band displacements.

In the physical mixture spectrum, the vibrational bands have the same displacement as the raw materials. Thus, three bands appear in the region of 3500 to 3000 cm⁻¹, which correspond to the O—H and N—H stretch of quercetin and arginine. The C═C vibrational band of aromatics at 1605 cm⁻¹ in the quercetin spectrum remains unchanged. The stretches corresponding to the C═O vibration of the carbonyl group of arginine are observed at 1708 and 1678 cm⁻¹. Since the vibrational bands of the physical mixture present the same shift of the raw materials, it is confirmed that it is indeed a physical mixture because there are no intermolecular interactions.

The spectrum of the QCT:ARG 1:8 NSF presents a widening and loss of definition of the vibrational bands, which confirms the amorphous character of the sample. Similarly, important shifts of the vibrational bands are observed. The bands of O—H and N—H vibrations observed at 3396, 3344 and 3265 cm⁻¹ in the spectra of quercetin and arginine, are joined in a single poorly-defined band with a maximum at 3336 cm⁻¹. The bands of the carbonyl groups observed at 1660 cm⁻¹ in quercetin and 1708 and 1678 cm⁻¹ in arginine, join in a wide and poorly-defined band with a maximum at 1640 cm⁻¹. The vibration band of the C═C bond of quercetin at 1605 cm⁻¹ undergoes a significant shift in the spectrum of the QCT:ARG 1:8 co-amorphous form, overlapping with the wide signal of the carbonyl groups at 1640 cm⁻¹. Based on the above, it can be concluded that the QCT:ARG NSF with stoichiometry 1:8 is co-amorphous.

### Thermal Analysis of the QCT:ARG 1:8 NSF

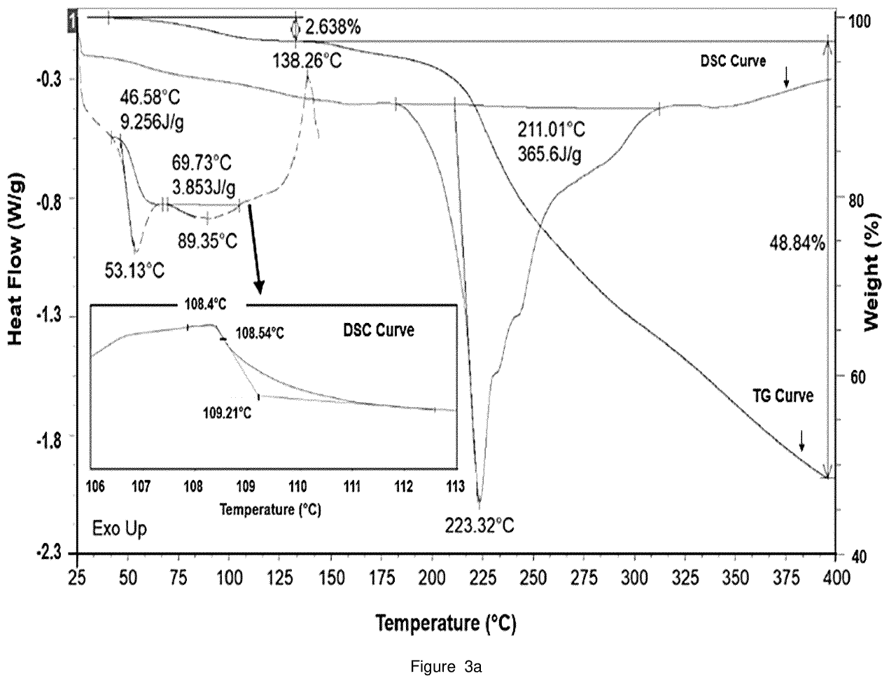

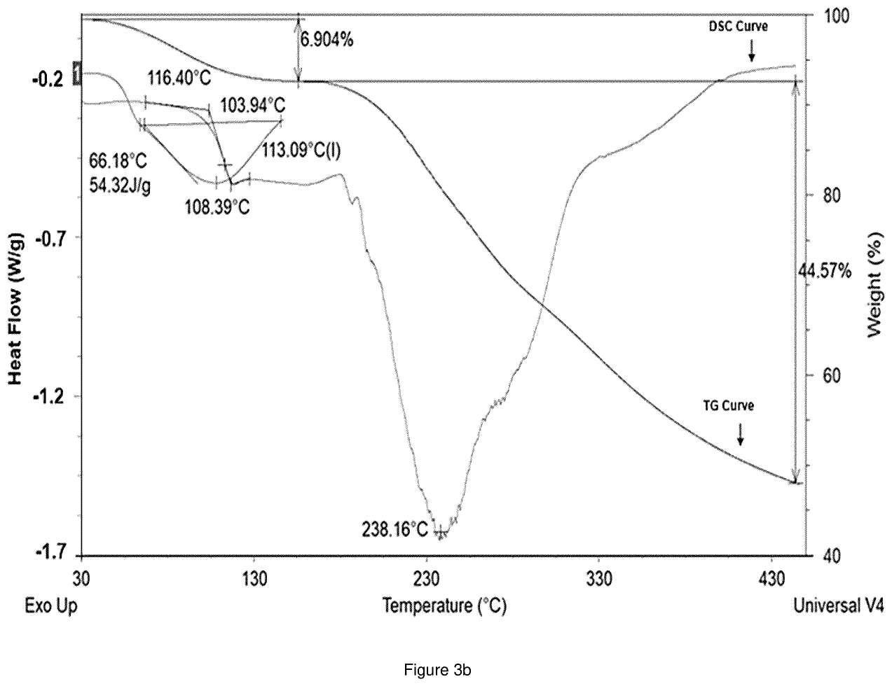

The thermal analysis (DSC and TGA) of the QCT:ARG 1:8 co-amorphous form is shown in FIG. 3a. In the first heating (dashed lines) two endothermic events can be observed at 53.13° C. and 89.35° C., the latter with a weight loss of 2.63%, which can be assigned to a phase change and a dehydration process of the sample. Based on the lost percentage, it can be determined that one mole of water is lost (calc. 2.13%). After desolvation, a glass transition is observed, starting at 108.4° C. Finally, at approximately 130° C., an exothermic event is observed that can be attributed to a crystallization. In the second heating no thermal event below 200° C. is shown. Subsequently, the decomposition point of the sample is observed, in which a weight loss of almost 50% occurs. Based on the above, and also considering the presence of a single glass transition, it is concluded that QCT:ARG 1:8 is a co-amorphous hydrate of quercetin:arginine.

### Example 2. New Solid Forms of Quercetin:Arginine (QCT:ARG) 1:3 to 1:9

New co-amorphous solid forms of QCT:ARG with a molar ratio of 1:3 to 1:9 were also obtained by rapid evaporation using water as a solvent. These samples were analyzed by X-ray powder diffraction and FT-IR ATR. It should be added that by using lower ratios of quercetin to arginine (1:2 or 1:1) it was impossible to completely solubilize quercetin in water for the rapid evaporation of the solvent.

Physical Analysis of NSF QCT:ARG with Molar Ratios 1:3 to 1:9

Initially, all samples had an orange color and were perfectly dry solids. It was observed that the orange color became more intense as the stoichiometry of the mixture increased. After 24 hours of exposure to the environment, those NSF with stoichiometries where arginine is in ratios greater than 4 moles, showed a higher hygroscopicity. The QCT:ARG 1:4 NSF was slightly compact and was not very hydrated. The QCT:ARG 1:3 NSF was observed as a dry and manageable solid with no signs of hydration.

### Thermal Analysis of the QCT:ARG 1:3 NSF

The thermal analysis (DSC and TGA) of the QCT:ARG 1:3 co-amorphous form is shown in FIG. 3b. In the first heating, two endothermic events can be observed at 66.18° C. and 108.39° C., the latter with a weight loss. After desolvation, a glass transition is observed starting at 108° C. In the second heating, no thermal event below 200° C. is shown. Subsequently, the decomposition point of the sample is observed at 238° C., in which a weight loss of almost 50% occurs. Based on the above, and also considering the presence of a single glass transition, it is concluded that this NSF is a co-amorphous quercetin:arginine hydrate.

### X-Ray Powder Diffraction of NSF QCT:ARG 1:3 to 1:9

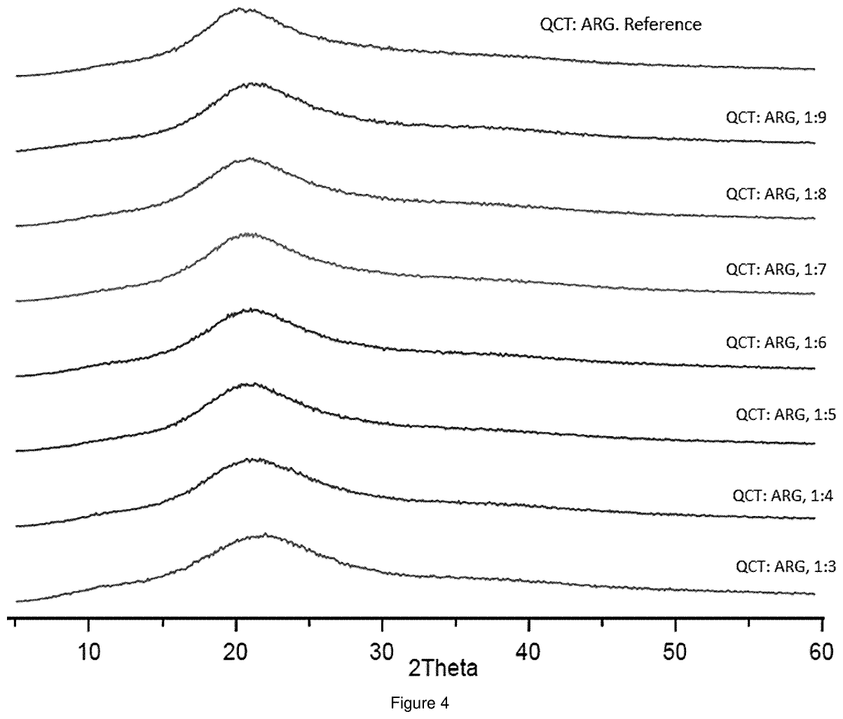

FIG. 4 shows the diffractograms obtained for these seven NSF. In all cases, the characteristic diffraction halo of amorphous solids is observed, and they have the same shape as the diffraction halo observed for the QCT:ARG 1:8 NSF.

### FT-IR ATR Spectroscopy of NSF QCT:ARG 1:3 to 1:9

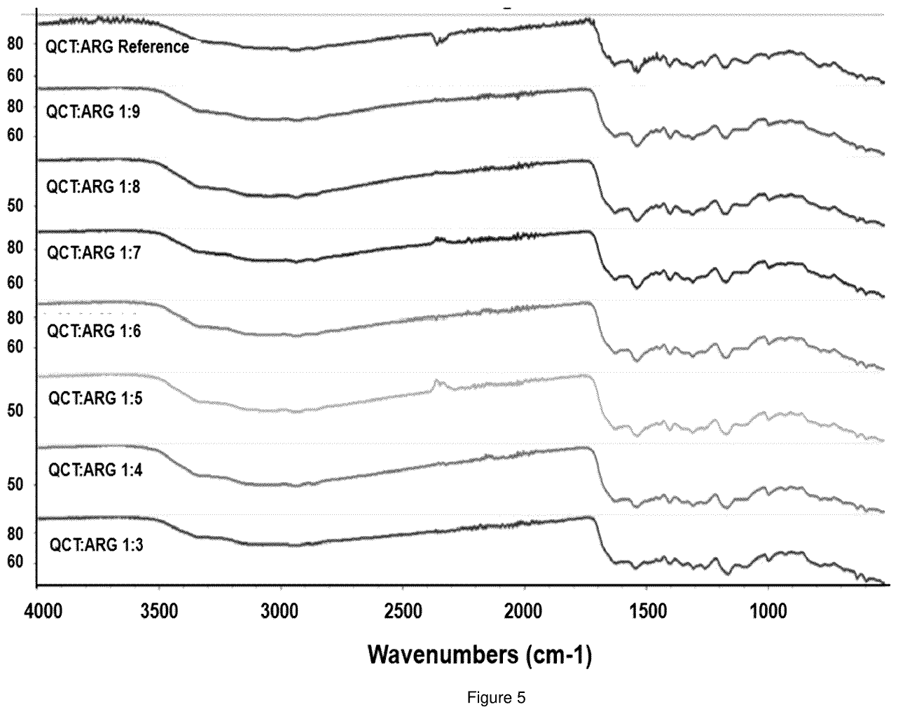

FIG. 5 shows a widening and loss of definition of the vibrational bands, which confirms the amorphous nature of all these samples. In addition, the pattern of the bands in very similar among them and also with respect to the IR spectrum of the co-amorphous form QCT:ARG 1:8, thereby concluding that all these samples maintain a very similar pattern of interactions between quercetin and arginine.

### Example 3. New Co-Amorphous Solid Form of Fisetin:Arginine (NSF FST:ARG) 1:8

A new co-amorphous solid form of Fisetin:Arginine (FST:ARG) with a ratio 1:8 was obtained by the method of rapid evaporation of water.

### Physical Analysis of the FST:ARG 1:8 NSF

The obtained FST:ARG NSF with stoichiometry 1:8 is a dark orange solid.

### X-Ray Powder Diffraction of FST:ARG 1:8 NSF

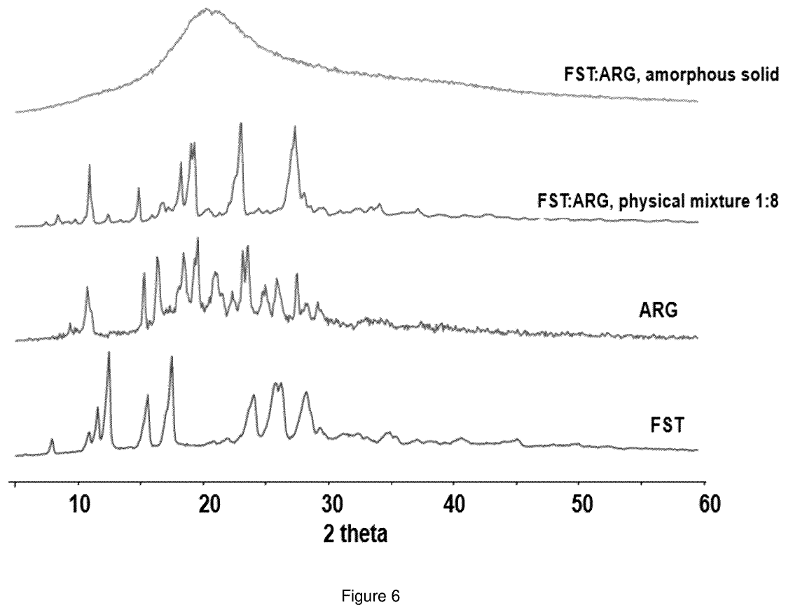

FIG. 6 shows the X-ray powder diffractogram of the raw materials, the physical mixture of fisetin and arginine, as well as the FST:ARG 1:8 NSF. The physical mixture shows a pattern of diffraction peaks similar to that of arginine, while the diffraction pattern of fisetin is observed as very small peaks. This can be attributed to the fact that fisetin is in a very low molar ratio to arginine. In contrast, the diffractogram of the solid FST:ARG 1:8 shows a diffuse halo without diffraction peaks characteristic of an amorphous solid compound. This, together with the color of the solid obtained, allows us to conclude that it is an amorphous NSF.

### FT-IR Spectroscopy of the FST:ARG 1:8 NSF.

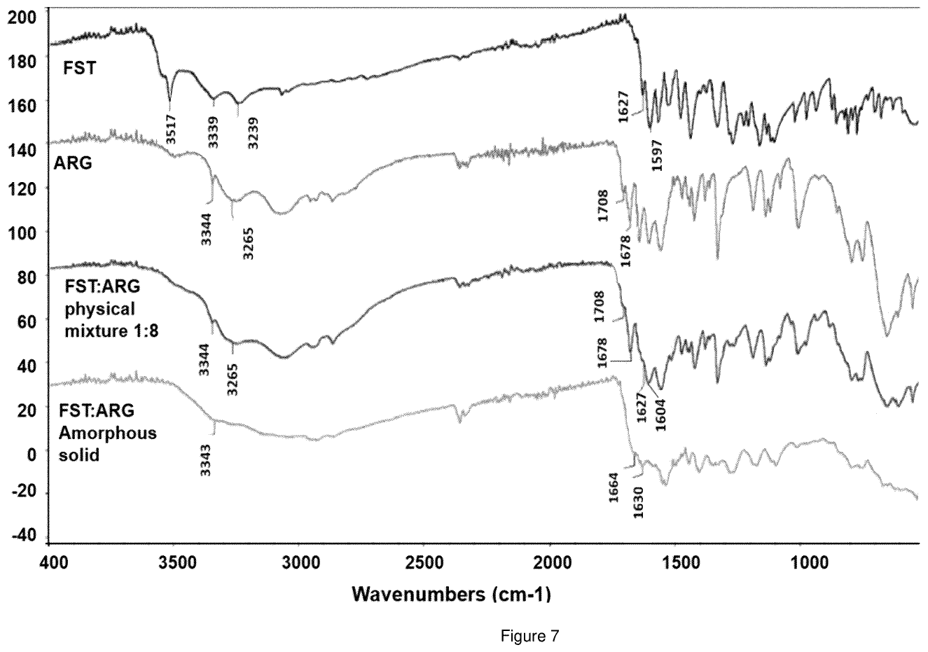

FIG. 7 shows the spectra of FT-IR fisetin, arginine, FST:ARG 1:8 NSF, and the physical mixture of fisetin and arginine.

In the spectrum of physical mixture, the vibrational bands of the O—H and N—H bonds (3200 to 2250 cm⁻¹) and of the carbonyls (1978 and 1708 cm⁻¹) that appear in the arginine spectrum do not undergo any shift changes. The vibration bands of fisetin do not appear, possibly due to the stoichiometric ratio in which fisetin is found with respect to arginine. This confirms that no intermolecular interactions between fisetin and arginine are formed in this sample.

The FT-IR spectrum for the FST:ARG 1:8 NSF shows wide, poorly defined bands commonly observed in amorphous forms. Similarly, important band shifts are observed, for example, the bands assigned to the O—H and N—H stretching of fisetin (3517, 3339 and 3239 cm⁻¹) and arginine (3344 and 3265 cm⁻¹) are joined in a single wide band with a maximum of 3343 cm-1. In the region of the carbonyl groups at 1627 cm⁻¹ in fisetin and at 1708 and 1678 cm⁻¹ in arginine, the bands combine in a single broad and poorly defined band with a maximum at 1630 cm-1. Based on these shiftings it can be concluded that fisetin and arginine maintain intermolecular interactions in the solid state.

### Thermal Analysis of FST:ARG 1:8 NSF.

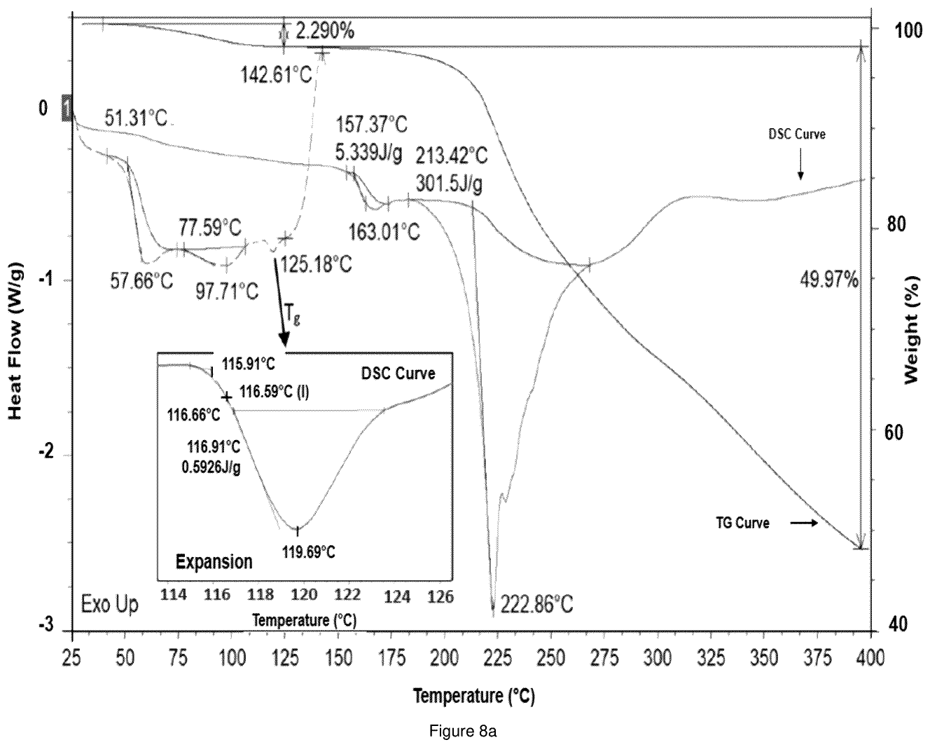

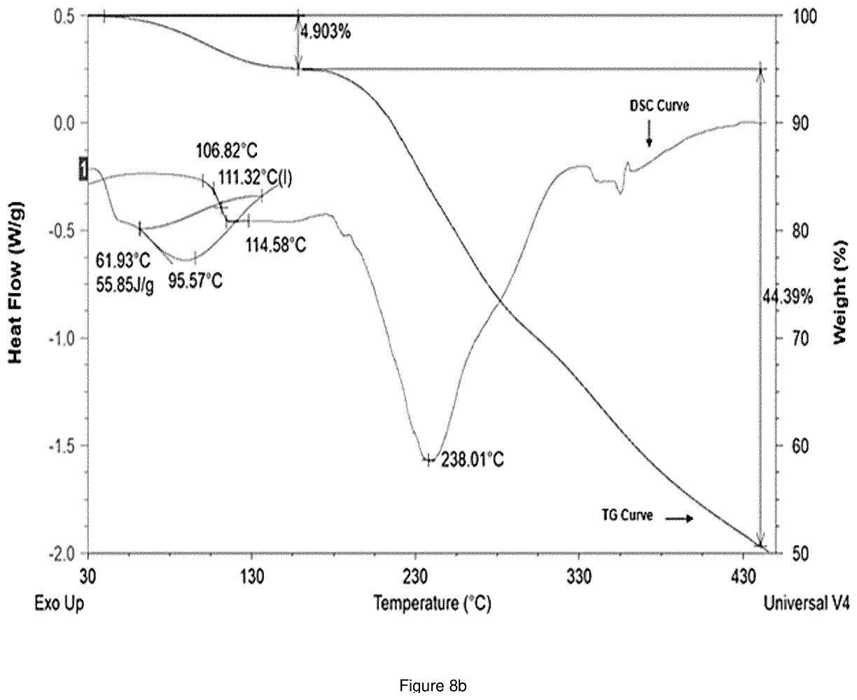

FIG. 8a shows the DSC and TGA thermogram. The initial heating to 150° C. (thermogram in dashed lines) shows several thermal events. The first events correspond to a possible phase change (57.66° C.) followed by dehydration (97.71° C.) with a weight loss of 2.29% corresponding to one mole of water (cal. 2.17%), which confirms the preparation of a co-amorphous hydrate. Subsequently, a glass transition with structural relaxation is observed at a starting temperature of 77.59° C. and a midpoint of 97.71° C., which confirms the amorphous nature of the sample. Also, the presence of a single glass transition indicates that it is a co-amorphous solid and not a physical mixture. The solid begins to crystallize at 125.18° C. In the second heating (solid line) no thermal event is observed until it reaches 157° C., where an endothermic event is observed without weight loss, corresponding to a phase change of the solid prior to the endothermic decomposition event observed at 213.42° C. (with a maximum at 222.86° C.) and has a weight loss of almost 50%. Based on the above it can be concluded that this NSF is a co-amorphous FST:ARG 1:8 hydrate.

### Example 4a. New Co-Amorphous Solid Forms of Fisetin:Arginine (FST:ARG) 1:3 to 1:10

New co-amorphous solid forms of FST:ARG were also obtained with molar ratios 1:3 to 1:10 by rapid evaporation using water as a solvent. These samples were analyzed by X-ray powder diffraction and FT-IR ATR. When using lower ratios (1:1 and 1:2) it was not possible to completely solubilize the fisetin in water to perform rapid solvent evaporation.

### Physical Analysis of the NSF FST:ARG 1:3 to 1:10

All NSF were dark orange and perfectly dry solids, coinciding with the color and characteristics of the co-amorphous solid FIS:ARG 1:8. Initially, all NSF were perfectly dry and manageable solids. After 24 hours of exposure to the environment, it was observed that those NSF with stoichiometries where arginine is found in ratios greater than 4 moles, show a high hygroscopicity. The higher the molar ratio of arginine, the more hygroscopic is the sample. NSF 1:4 was observed slightly hydrated, whereas FST:ARG 1:3 NSF remains longer stable.

### Thermal Analysis of FST:ARG 1:3 NSF.

The thermal analysis (DSC and TGA) of the FST:ARG 1:3 co-amorphous form is shown in FIG. 8b. Initial heating to 150° C. shows several thermal events. The first events correspond to a possible phase change (61.93° C.) followed by dehydration (95.57° C.) with a weight loss, which confirms that an amorphous co-hydrate was obtained. Subsequently, a glass transition with structural relaxation is observed at an initial temperature of 106.82° C. and a midpoint of 95.57° C., which confirms the amorphous nature of the sample. Furthermore, the presence of a single glass transition indicates that it is a co-amorphous solid and not a physical mixture. The solid begins to crystallize at 111.32° C. In the second heating (solid line) no thermal event is observed prior to the endothermic decomposition even observed above 200° C. (with a maximum of 238.01° C.) and it has a weight loss of almost 50%. Based on the above, it can be concluded that this NSF is a co-amorphous FST:ARG 1:3 hydrate.

### X-Ray Powder Diffraction of NSF FST:ARG 1:3 to 1:10

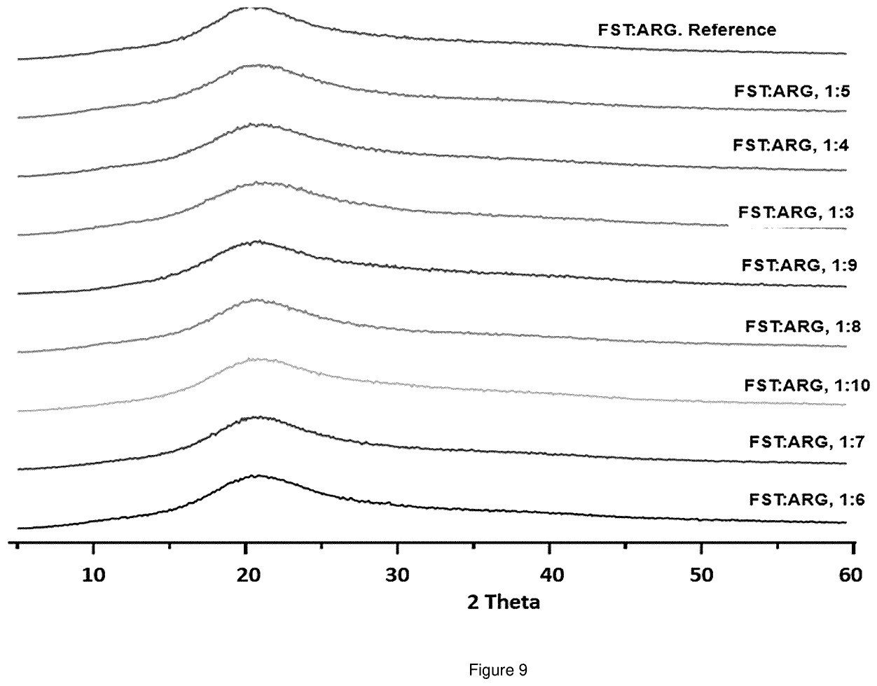

FIG. 9 shows the diffractograms obtained for each NSF and the diffractogram FST:ARG 1:8 NSF is included as reference. All solid forms show the diffraction halo characteristic of amorphous solids, and they have the same shape as the diffraction halo observed for FST:ARG 1:8 NSF.

### FT-IR ATR Spectroscopy Studies of the NSF FST:ARG 1:3 to 1:10

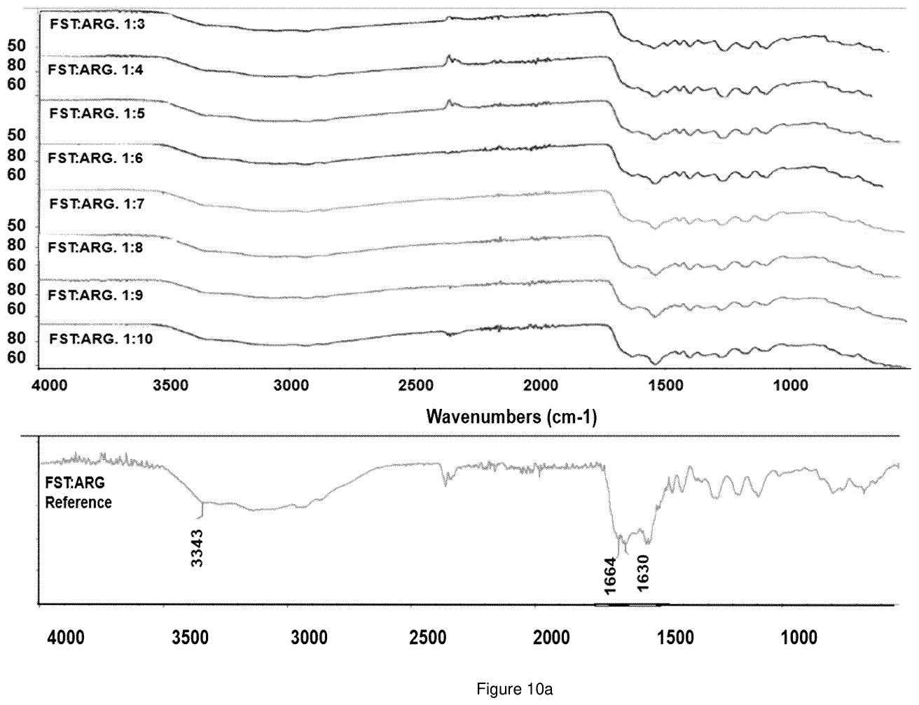

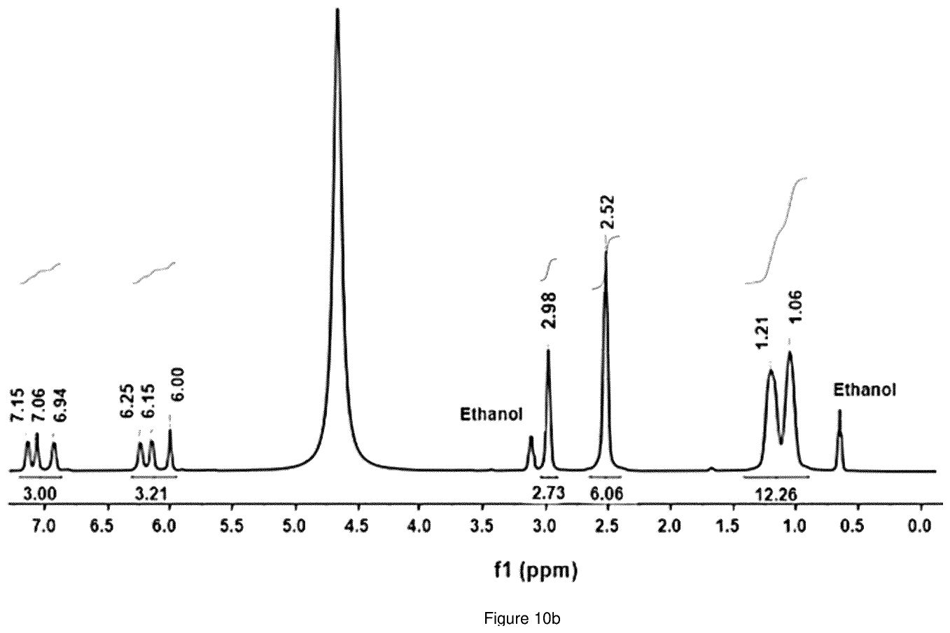

FIG. 10a shows a widening and loss of definition of the vibrational bands which confirms the amorphous nature of all these samples. In addition, the pattern of the bands in very similar among them, and also with respect to the IR spectrum of the co-amorphous solid (FST:ARG 1:8) used as reference. It is concluded that all samples maintain a pattern of very similar interactions between FST and ARG.

### Example 4b. New Co-Amorphous Solvate FST:ARG:Ethanol 1:3:1 Obtained by Rapid Evaporation of Ethanol/Water

Arginine was initially dissolved in a round-bottom flask with a minimum amount of water. Fisetin and 30 mL of ethanol were subsequently added to the flask, until the mixture was completely dissolved, and was rotavaporated to dryness. The obtained solid was analyzed by NMR¹H.

The NMR¹H spectrum in D₂O of FIG. 10b shows six peaks between 6 and 7.2 ppm that were assigned to the hydrogens of the fisetin aromatic rings, these six peaks show an integral for 6 hydrogens. A signal integrating for 3 hydrogens appears at 2.98 ppm that was assigned to the hydrogen of the alpha carbon arginine, while at 2.52 ppm another signal was observed at 2.52 ppm which was assigned to the CH₂—NH hydrogens of the guanidinium fragment. A pair of multiplets at 1.21 and 1.06 ppm were assigned to the CH₂ of the arginine chain. The calculation of the integral shows that arginine is in a 1:3 stoichiometric fisetin:arginine ratio. A triplet and a quartet assigned to an ethanol molecule are observed at 0.65 and 3.11 ppm. This confirms that the obtained NSF is a co-amorphous FST:ARG:ethanol 1:3:1 solvate.

### Example 5a. New Solid Co-Amorphous Forms of Rutin:Arginine (RTN:ARG) 1:1 to 1:4

NSF of rutin and arginine were prepared with different ratios 1:1, 1:2, 1:3, 1:4 using the rapid evaporation method. A mixture of rutin and arginine and 25 ml of water were placed in a 100 mL balloon flask. The mixtures were placed in the rotary evaporator and were immersed in a water bath at 80° C., leaving them in rotation until the mixture was completely dissolved. Subsequently, the pressure of the rotary evaporator was decreased until the solution was completely evaporated, obtaining a dry and foamy solid.

### Physical Analysis of NSF RTN:ARG 1:1 to 1:4

The solids obtained by rapid evaporation with ratios 1:2, 1:3 and 1:4, showed a coloration change from light yellow to bright orange. In contrast, for the solid RTN:ARG 1:1 no color change was observed and the sample only becomes brighter and takes the form of flakes.

When handling the samples, it was noted that the 1:2 and 1:4 stoichiometries began to stick together in the vial the day after they were obtained. In contrast, the RTN:ARG 1:3 sample remains a dry solid at 24 hrs. Based on the above, we conclude that the 1:3 RTN:ARG mixture is the stoichiometry more stable than the previous ones.

### X-Ray Powder Diffraction of NSF RTN:ARG 1:1 to 1:4

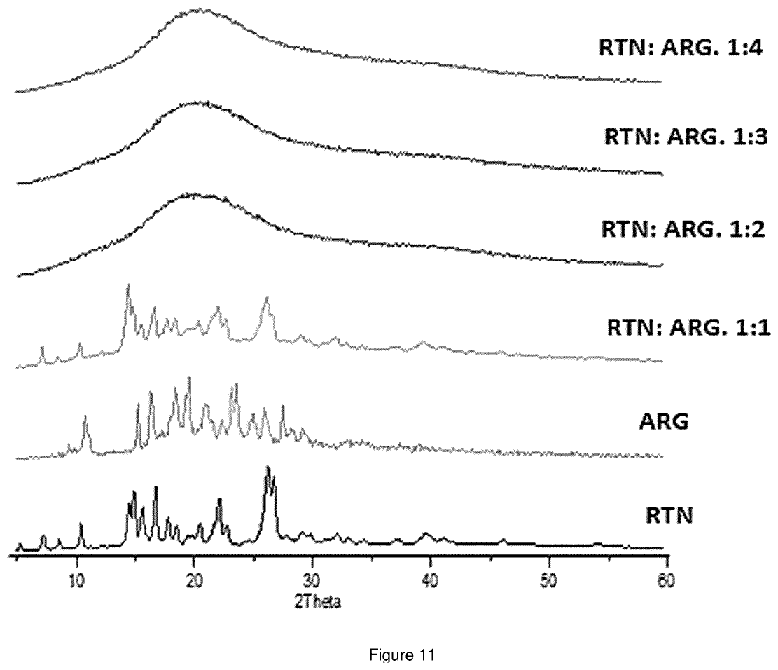

X-ray diffraction spectra were obtained for RTN:ARG NSF with ratios 1:1, 1:2, 1:3 and 1:4, and were compared with the raw materials. FIG. 11 shows a halo characteristic of an amorphous solid compound in the spectra of the NSF 1:2, 1:3 and 1:4, whereas the spectra of the raw materials rutin and arginine show the presence of diffraction peaks. On the other hand, the NSF 1:1 shows the peaks corresponding to rutin, which is in excess.

### FT-IR Spectroscopy of NSF RTN:ARG 1:1 to 1:4

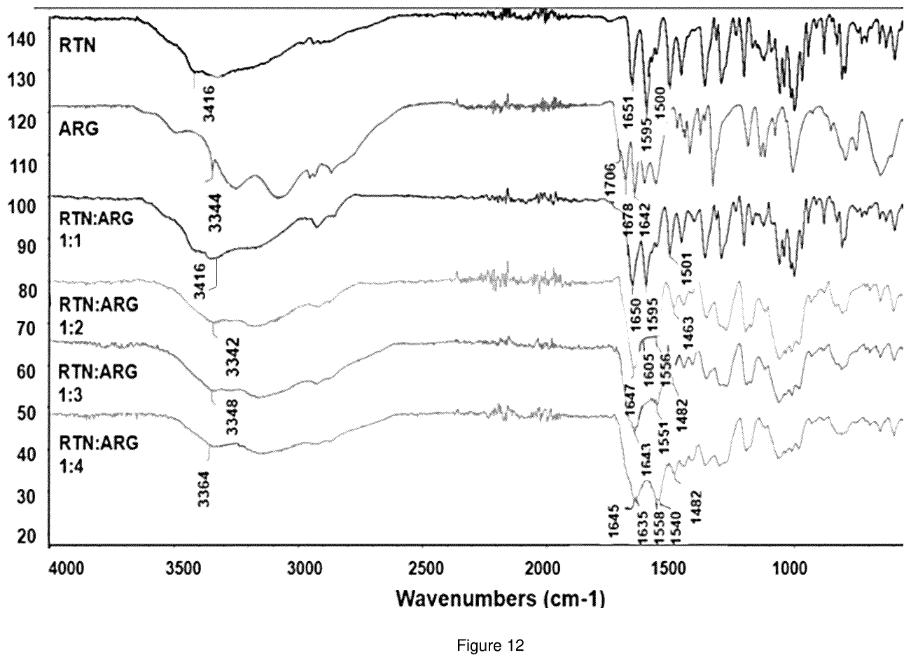

FIG. 12 shows the FT-IR spectra of raw materials and NSF of RTN:ARG 1:1, 1:2, 1:3, 1:4.

The spectrum of rutin shows at 3416 cm⁻¹ the characteristic broad stretching band of the hydroxyl groups; at 1651 cm-1 the characteristic band due to the stretching vibration of the C═O bond of the carbonyl group; and at 1595 and 1500 cm⁻¹ two bands of the stretching vibration of the C═C bond of the aromatic groups. For arginine, the most important bands are a small well-defined band at 3344 cm⁻¹ that was assigned to the stretching of the NH₂ group, at 1706 cm⁻¹ a small shoulder assigned to the stretching of the C═O bond of the carboxyl group, and two more bands at 1678 and 1641 cm⁻¹ assigned to the asymmetric bending vibrations of group NH₂ and the N—H bending vibration of the guanidinium ion, confirming that arginine is present in its zwitterionic form.

The IR spectrum of the RTN:ARG 1:1 NSF shows a band pattern very similar to that of rutin, with shifts of the minimum vibrational bands (with differences between 1 and 2 cm⁻¹). Based on this, it is assumed that rutin is found in excess, as observed in the X-ray powder diffractogram.

For the RTN:ARG 1:2 and 1:3 mixtures, fully widened vibrational bands are observed, which confirm the amorphous nature of these NSF. The band at 3416 cm⁻¹ corresponding to rutin and the well-defined band at 3344 cm⁻¹ corresponding to arginine, disappear and bind in a single broad band at 3342 and 3348 cm⁻¹, indicating that the hydroxyl and amino groups form new intermolecular interactions. Similarly, the vibrational bands of the carbonyl groups and aromatic carbons at 1651 and 1595 cm⁻¹ present in the rutin sample, and the bands assigned to the carbonyl, guanidinium and amino groups of arginine (1706, 1678 and 1642 cm⁻¹ respectively), join together in two wide bands with maximums at 1647 and 1556 cm⁻¹ in RTN:ARG 1:2, and 1643 and 1551 cm⁻¹ in RTN:ARG 1:3, indicating that these functional groups establish new intermolecular interactions. However, in the spectrum of RTN:ARG 1:2 a small band is observed at 1605 cm⁻¹, which is assigned to the stretching of the aromatic C═C bond observed at 1595 cm⁻¹ in RTN. Based on this, we assume that a small amount of unreacted RTN is still found in excess in the stoichiometry 1:2. Finally, for the IR spectrum of RTN:ARG 1:4, the only difference with the RTN:ARG 1:3 spectrum is that the band at 1551 cm⁻¹ increases in intensity.

### Thermal Analysis of NSF RTN:ARG 1:2, 1:3 and 1:4

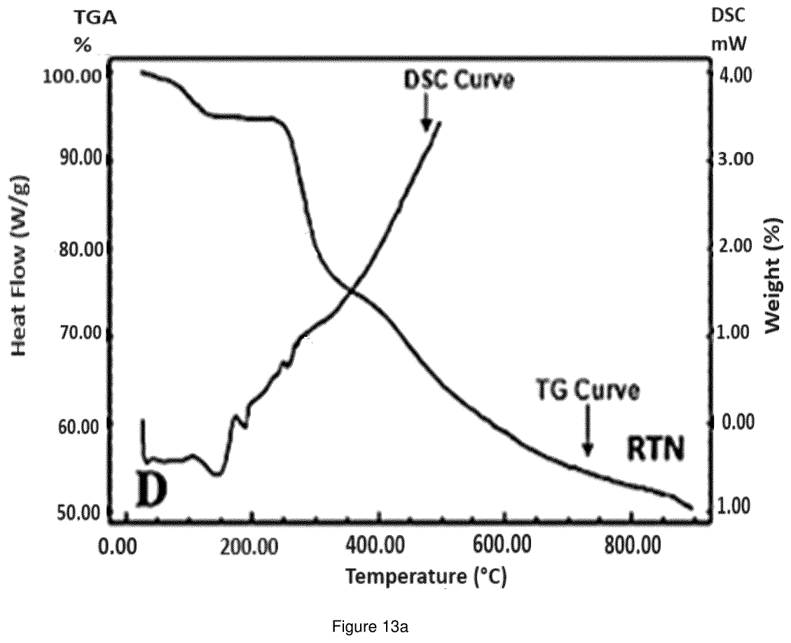

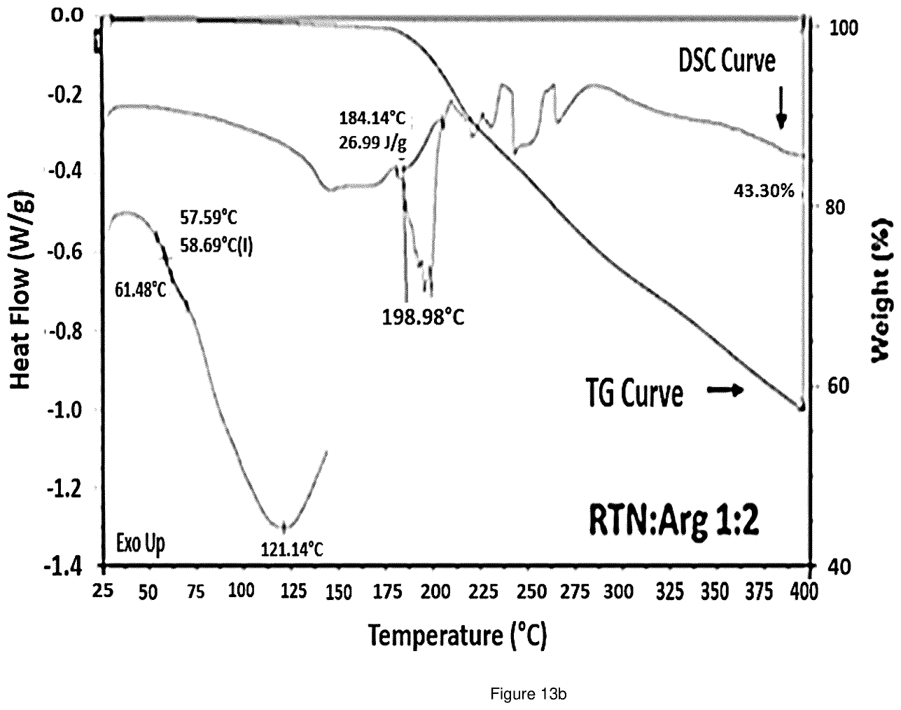

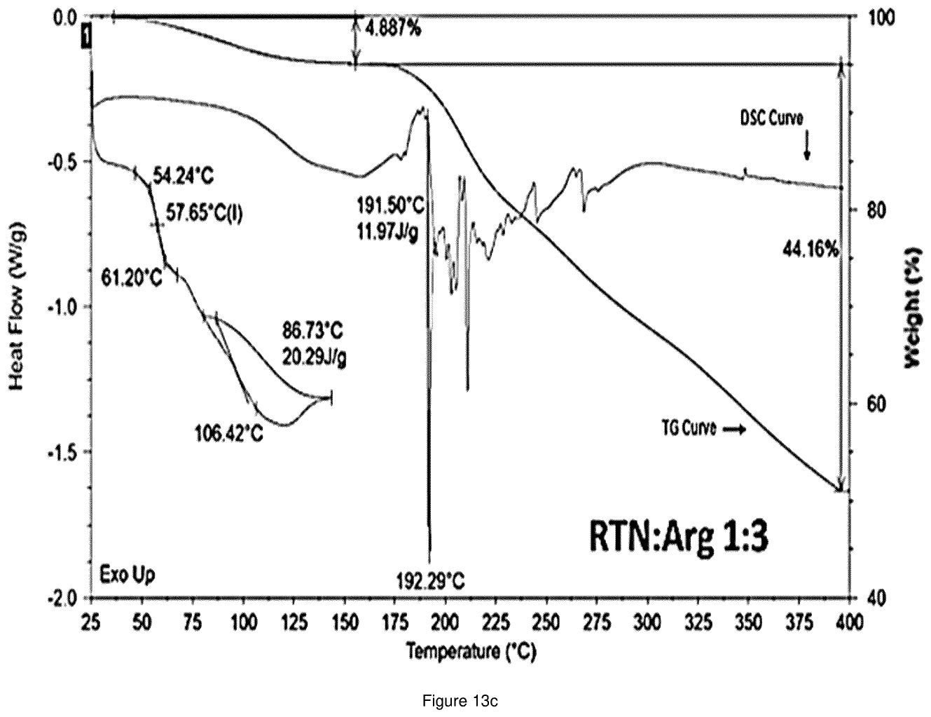

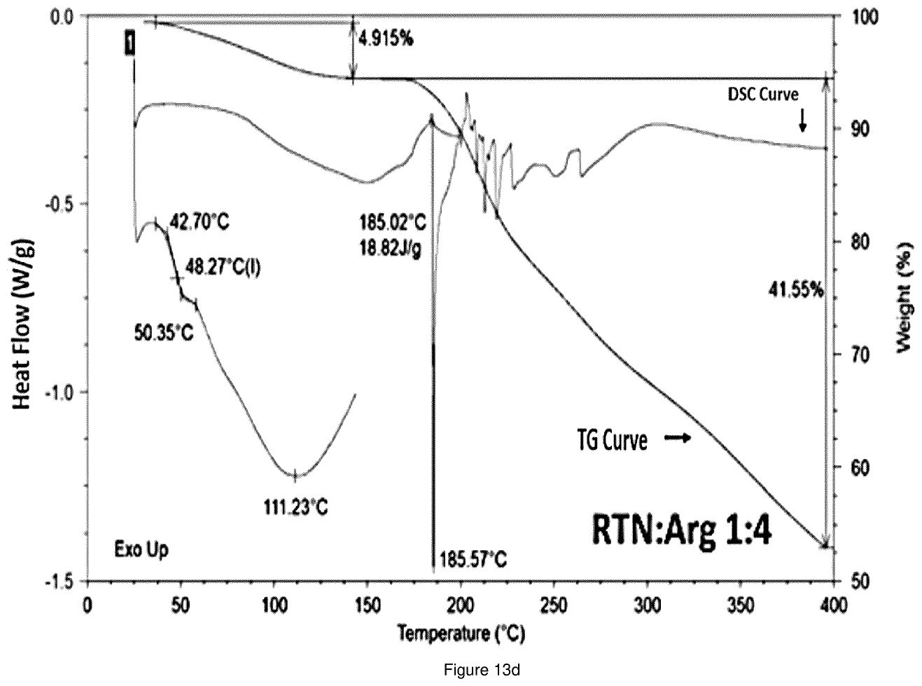

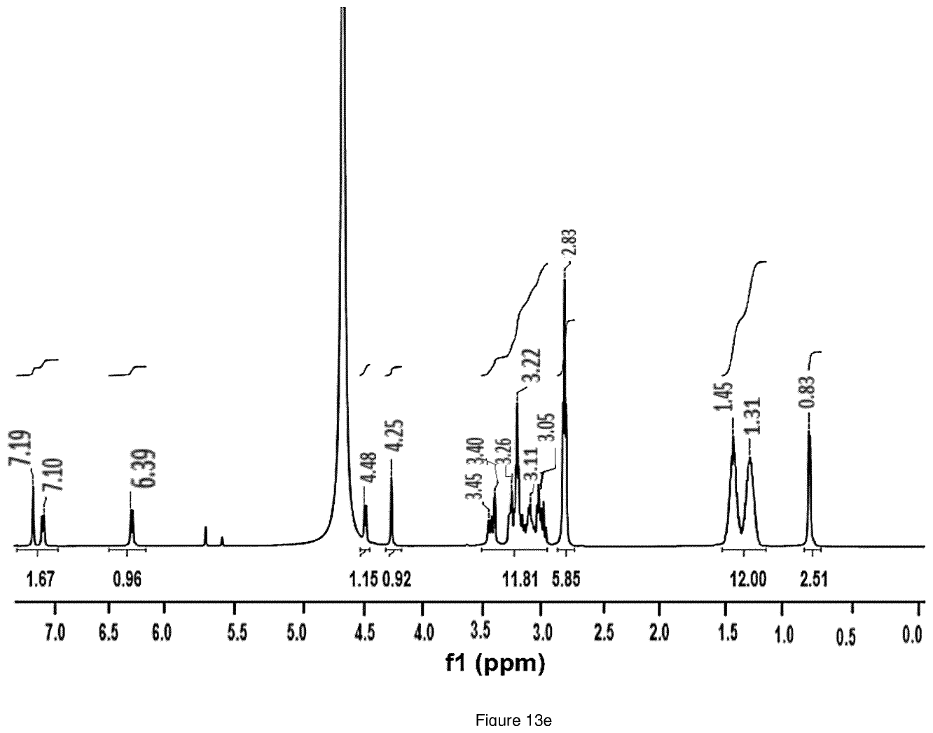

FIGS. 13a-13d shows DSC and TGA thermograms of RTN (FIG. 13a), RTN:ARG 1:2 NSF (FIG. 13b), RTN:ARG 1:3 NSF (FIG. 13c) and RTN:ARG NSF 1:4 (FIG. 13d). The three NSF show a very similar thermal character among them. In the first heating curve (slim lines) a thermal event at 58, 57 and 48° C. can be observed in the three stoichiometries, which can probably be attributed to a glass transition. It should be noted that in the solid 1:2 this glass transition is not as evident as in the stoichiometries 1:3 and 1:4, thus it can be assumed that the formation of a co-amorphous solid in the stoichiometry 1:2 is not complete, leaving unreacted rutin, as observed in the FT-IR spectra. After the glass transition, an endothermic event can be observed at 121° C. for the solid with a 1:2 ratio without weight loss in the TGA, which could be attributed to the phase changes observed for rutin. For stoichiometries 1:3 and 1:4, endothermic events were observed at 106 and 111° C. with losses of 4.88 and 4.9% in the TGA that were assigned to the loss of 3 and 3.5 moles of water respectively (cal. for 1:3 4.69% and 1:4 5.43). In the second heating (solid lines) no event is observed until 198, 192 and 185° C., where the decomposition/sublimation of the phase begins.

### Example 5b. New Solid Co-Amorphous Solvate of RTN:ARG:Ethanol 1:3:1 Obtained by Rapid Evaporation of Ethanol/Water

Arginine was initially dissolved in a round-bottom flask with a minimum amount of water. Rutin and 30 mL of ethanol were subsequently added to the flask, until the mixture was completely dissolved, and was rotavaporated to dryness. The obtained solid was analyzed by NMR¹H.

The NMR ¹H spectrum in D₂O (FIG. 13e) shows signals at 7.19, 7.10 and 6.37 ppm assigned to the hydrogens of the phenolic ring. These three signals integrate for three hydrogens. A set of signals between 3 to 3.5 ppm were assigned to the hydrogens of the rutin sugars and the hydrogens alpha to the carbonyl. At 2.83 ppm, a triplet integrating for 6 is assigned to methylene hydrogens bound to guanidinium of arginine. At 1.3 and 1.45 ppm two multiplets were observed integrating for 12 hydrogens and were assigned to the carbons of the aliphatic arginine chain. A doublet at 0.82 ppm was assigned to the methyl of the rutin sugar. The presence of ethanol was evidenced by a small triplet of 0.70 ppm and was confirmed by its DSC/TGA thermogram where a mass loss is observed at 80-110° C. Based on the above, it can be concluded that this NSF is a co-amorphous RTN:ARG:ethanol 1:3:1 solvate.

### Example 6. New Co-Amorphous Solid Forms of Genistein:Arginine (GTN:ARG) 1:3 to 1:10 by Rapid Evaporation

NSF of GTN:ARG with different stoichiometric ratios were prepared using the method of solvent rapid evaporation.

Firstly, the mixture GTN:ARG 1:3 was placed in a balloon flask and 30 ml of water were added. This mixture was placed in the rotary evaporator at 80° C. and was left in rotation until it was dissolved. However, after 20 min of agitation, it did not dissolve. Thus, the amount of arginine was increased until obtaining a stoichiometric ratio of 1:4, leaving it again in rotation and submerging it in the bath of the rotavap. The mixture was not dissolved, thus the stoichiometry of the mixture was progressively increased to 1:5, 1:6, 1:7, 1:8 and 1:9. For the latter, the mixture was completely dissolved. Once dissolved, the water began to evaporate, gradually decreasing the pressure until reaching almost dryness, where the pressure was reduced as much as possible. However, although it was left to dryness, a highly viscous amber liquid was obtained with a precipitate at the bottom, attributable to an excess of L-arginine.

To find the correct stoichiometry in which arginine was not in excess, the solvent rapid evaporation was carried out again starting with a 1:4 stoichiometry in 30 ml of water. For a matter of time, this mixture was left to rest overnight, observing the next morning that the contents of the mixture were completely dissolved. Once the 1:4 mixture was solubilized, the solvent was rapidly evaporated under the same conditions described above. However, after evaporation of the solvent, the amber viscous liquid was again obtained, but any precipitate was found.

### Example 7. New Amorphous Solid Forms of Genistein:Arginine (GTN:ARG) 1:3 to 1:10 Using the Slurry Technique

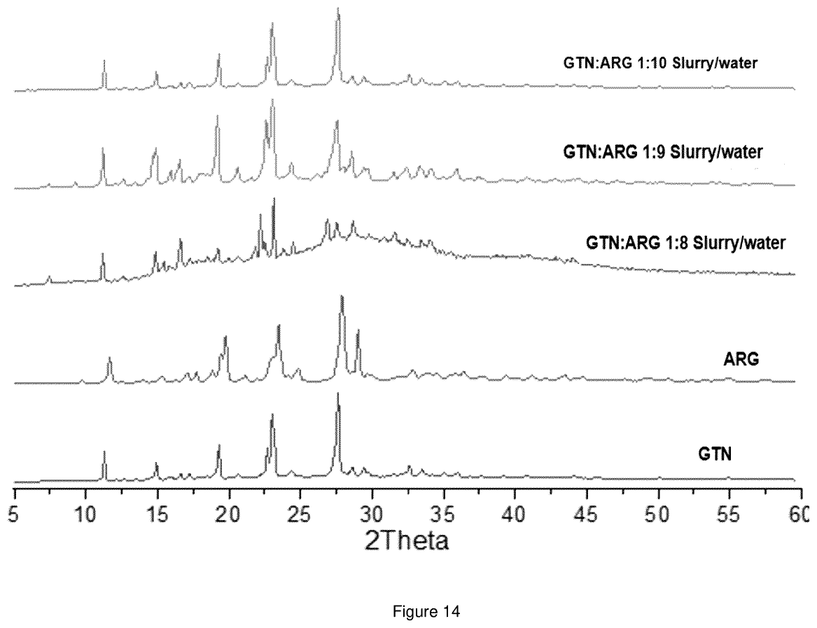

Since a dry, foamy solid could not be obtained by rapid evaporation (Example 6), the slurry technique was tried because it uses little water. Thus, in separate vials provided with magnetic stirring, the mixtures GTN:ARG 1:8, 1:9 and 1:10 were placed. 5 drops of water were added and left stirring for 10 min. None of the vials achieved the complete dissolution of the mixture, leaving the typical slurry. After the slurry time, the vials were left to dry overnight. The resulting solids GTN:ARG 1:8, 1:9 and 1:10 were characterized by X-ray powder diffraction and the diffractograms were compared with those of the raw materials as shown in FIG. 14. All diffractograms corresponding to slurries 1:8, 1:9 and 1:10, show diffraction peaks that correspond to the arginine that remains unreacted.

Subsequently, slurry under heating was carried out. Thus, to the mixtures GTN:ARG 1:9 and 1:10, 4 drops of water were added again along with a magnetic stirrer, and were placed on the hot griddle at 80° C. After 3 minutes, the mixtures GTN:ARG 1:9 and 1:10 acquired an intense orange coloration and were completely solubilized, leaving a transparent liquid without any precipitate. After 10 min of slurry, the solution was removed from the griddle and the vial was allowed to cool to room temperature. During the cooling it was observed that the solution quickly began to present transparent crystals corresponding to the excess of arginine. The crystallization was observed more intense in the mixture GEN:ARG 1:10, while in the solid with stoichiometry 1:9 a smaller amount of crystals was observed.

Based on the above, the stoichiometric ratio was reduced by performing two slurries with heating of mixtures GTN:ARG 1:6 and 1:3. The mixture of GTN:ARG 1:6 resulted in an intense orange liquid and completely solubilized, while the mixture GTN:ARG 1:3 was not completely solubilized. Once the vials were removed and then cooled, it was observed that in the 1:6 mixture it presented a small amount of arginine crystals.

Based on the above, an amount of arginine necessary to obtain a stoichiometric ratio 1:4 GTN:ARG was added to the mixture GTN:ARG 1:3 that did not dissolve, and hot slurry was carried out. After 2 minutes, the vial was completely solubilized leaving a transparent orange liquid. After cooling of the vial to room temperature, the presence of arginine crystals was not observed, concluding that the correct stoichiometry of the GTN:ARG NSF is 1:4, however, the sample remained as a highly viscous liquid.

### Example 8. New Co-Amorphous Solid Form GTN:ARG 1:4 Obtained by Dry Grinding and Wet Grinding

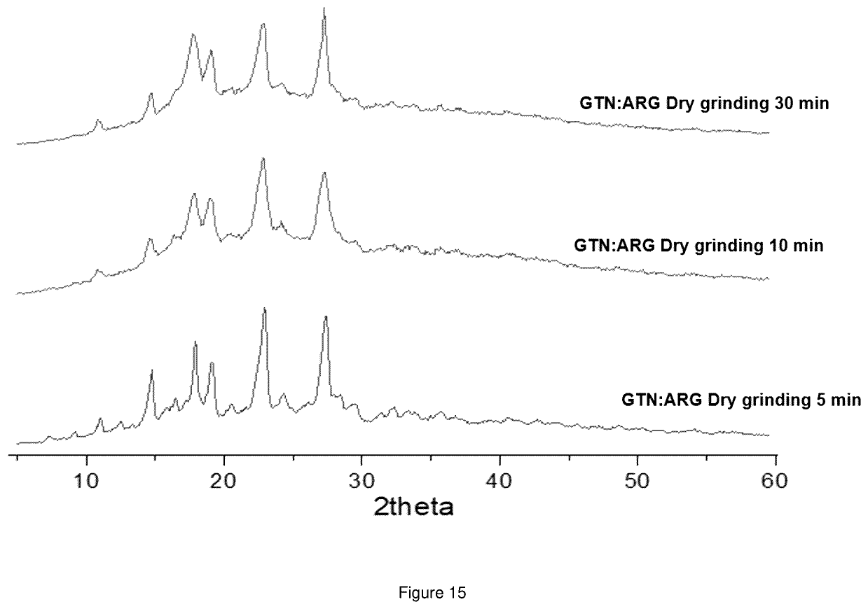

To evaluate techniques different from Examples 6 and 7, the preparation by dry grinding of the GTN-ARG 1:4 mixture was carried out. A mixture GTN:ARG 1:4 was placed in the mill container fitted with a grinding ball. The sample was ground for 5, 10 and 30 min at 30 Hz. At different times during grinding, the obtained powders were characterized by X-ray powder diffraction of as shown in FIG. 15. All ground samples presented diffraction peaks belonging to amorphous genistein that did not interact with arginine.

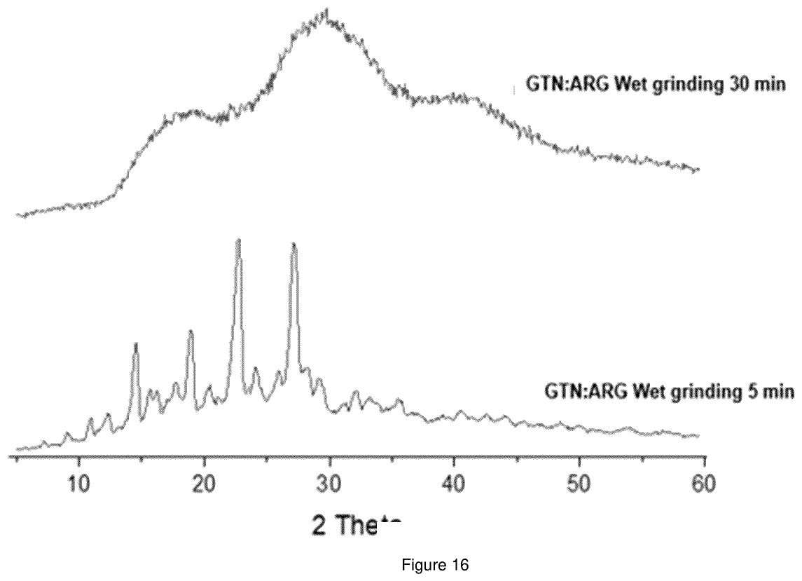

Subsequently, the GTN:ARG 1:4 mixture was ground with a few drops of water for 5 and 30 min. The resulting non-dry thick solids were left to dry overnight. The corresponding diffractograms are shown in FIG. 16.

The spectrum of the sample GTN:ARG 1:4 obtained by wet grinding at 5 min shows diffraction peaks corresponding to the raw materials that did not transform into an amorphous state. In contrast, when the mixture is subjected to the wet grinding process for 30 min, the mixture presented a complete amorphization of the raw materials. Based on the above, grinding was considered to be a possible methodology to obtain new amorphous solid forms of genistein.

### Example 9. Comparison of New Co-Amorphous Solid Forms of GTN:ARG 1:4 Obtained by Dry Grinding and Slurry

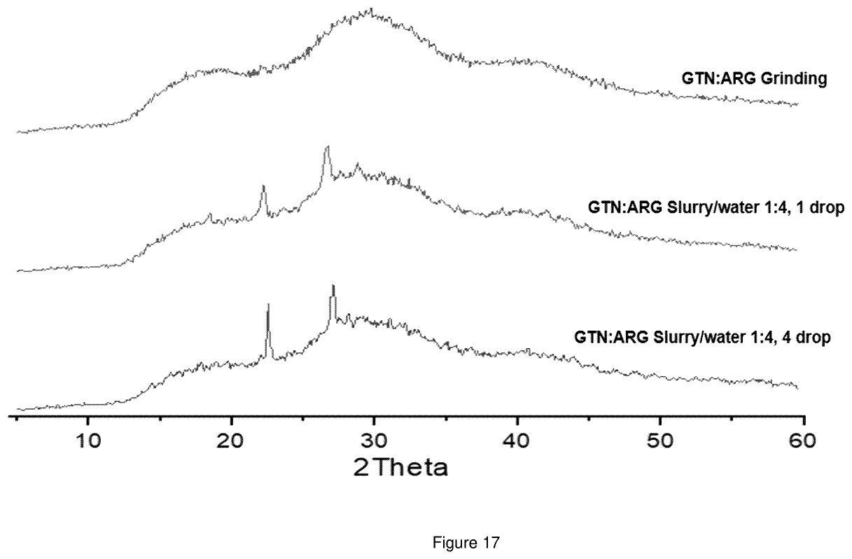

In an attempt to dry the viscous liquid obtained by slurry, the mixture GTN:ARG 1:4 was placed in two vials. One drop of water was added to one vial and four drops of water were added to the other vial. They were left under agitation and heating at 80° C., and immediately after the liquid was formed, it was transferred and extended to an aluminum tray leaving it to dry for one night. After such time, complete dryness was observed, obtaining a completely manageable solid which was characterized and compared with the solid obtained by wet grinding at 30 min (Example 8) as shown in the diffractograms of FIG. 17.

The samples obtained by slurry show a diffuse diffraction halo that presents three regions with maximums, which correspond to the halo observed in the wet grinding diffractogram. However, the presence of two diffraction peaks in the slurries pattern corresponding to the diffraction pattern of genistein, indicates that, although the slurry rendered the same solid form, there is also unreacted genistein.

### Example 10. Comparison of the New Co-Amorphous Solid Forms of GTN:ARG 1:4 Obtained by Rapid Evaporation of Water, Slurry in Water and Grinding in Water

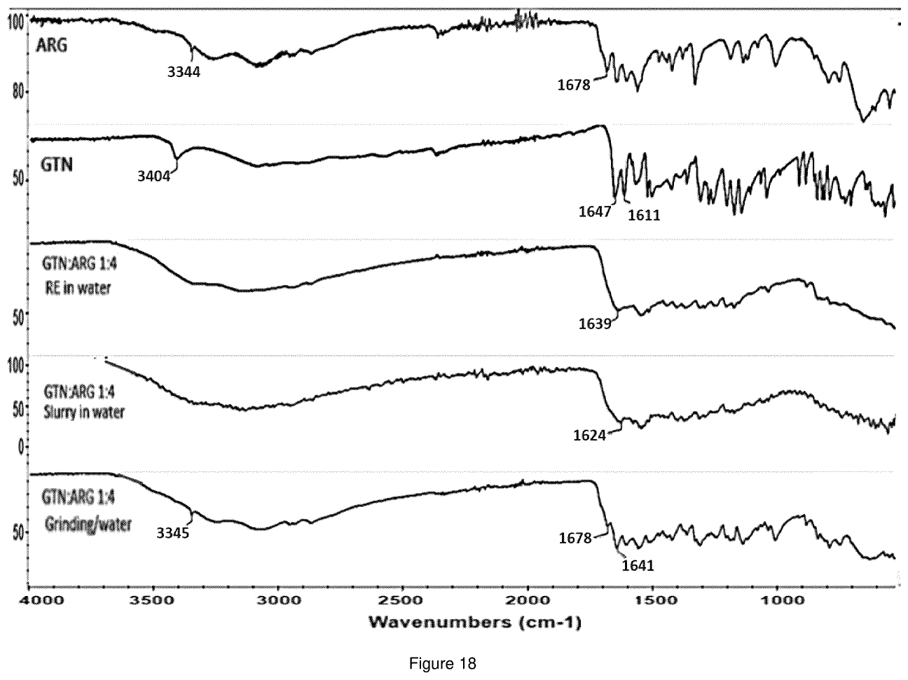

To corroborate the existence of intermolecular interactions between genistein and arginine, FT-IR spectroscopy studies were performed to the solid obtained by rapid evaporation of GTN:ARG 1:4 in water, the slurry in water of GTN:ARG 1:4 and the solid obtained by water grinding of GTN:ARG 1:4. These samples were compared with the GTN and ARG raw materials as shown in FIG. 18.

The FT-IR spectra of the raw materials show that the most important bands for arginine are a small and well-defined band at 3344 cm⁻¹ and a wide band at 1678 cm⁻¹ assigned to the stretching and bending vibrational bands of the ˜NH₂ bond. On the other hand, in the spectrum of genistein, a small well-defined vibrational band at 3404 cm⁻¹ is observed, which is attributed to the vibration of the O—H bond of genistein, as well as two bands at 1647 and 1611 cm⁻¹ that were assigned to the vibrations of C═O and C═C bonds of genistein.

In the FT-IR spectra of GTN:ARG 1:4 obtained by rapid evaporation (RE) of water and slurry in water, a pattern of similar vibrational bands is observed, concluding that these two methodologies allow obtaining the same type of solid. Additionally, the vibrational bands are less defined and wider, which is attributed to the process of amorphization of the samples. In addition, the bands observed at 3344 cm⁻¹ and 3404 cm⁻¹ for arginine and genistein disappear completely in the spectra of the solid obtained by rapid evaporation and slurry. This can be attributed to the fact that these functional groups could establish intermolecular interactions in those solids. Similarly, in both cases, it can be observed that the bands observed at 1678 cm⁻¹, 1647 cm⁻¹ and 1611 cm⁻¹, join in a single broad band at 1639 and 1624 cm⁻¹ in the spectra corresponding to rapid evaporation in water and slurry in water. This indicates that the formation of intermolecular interactions between genistein and arginine is favored in the semisolids obtained by these techniques.

In the FT-IR spectra of GTN:ARg 1:4 obtained by grinding in water, much more defined bands are observed than in the spectra of the semisolids of the ERD and the slurry. This could indicate the presence of crystalline residues in the sample, in addition a small band can be observed at 3344 cm⁻¹ that corresponds to the vibration of the —NH₂ bond of arginine, in the same way at 1678 cm⁻¹ and 1641 cm⁻¹ two bands assigned to the vibrations of the bonds —NH₂ and C—NH₂ can be observed, which are assigned to arginine and aromatic C═C vibrations of genistein, respectively. Based on this, it is estimated that there is a considerable amount of raw material without reacting and that it is a possible physical mixture.

### Example 11. New Co-Amorphous Solvate GTN:ARG:Ethanol 1:4:1 Obtained by Rapid Evaporation of Ethanol/Water

Since a semisolid was obtained using the technique of rapid evaporation in water, an alternative methodology was sought to obtain the GTN:ARG 1:4 NSF as a dry solid by rapid evaporation of ethanol/water. For the purpose, arginine was initially dissolved in a round-bottom flask with a minimum amount of water. Genistein and 30 mL of ethanol were subsequently added to the flask, until the mixture was completely dissolved, and was rotavaporated to dryness. When the solvent was completely evaporated, the formation of the foam typical of amorphous solids was observed, which could be perfectly dried and scraped to obtain the solid.

### Physical Analysis of the GTN:ARG 1:4 NSF Obtained by Rapid Evaporation of Ethanol/Water

The solid is a completely dry and manageable light-yellow powder.

### Thermal Analysis of GTN:ARG 1:4 NSF Obtained by Rapid Evaporation of Ethanol/Water

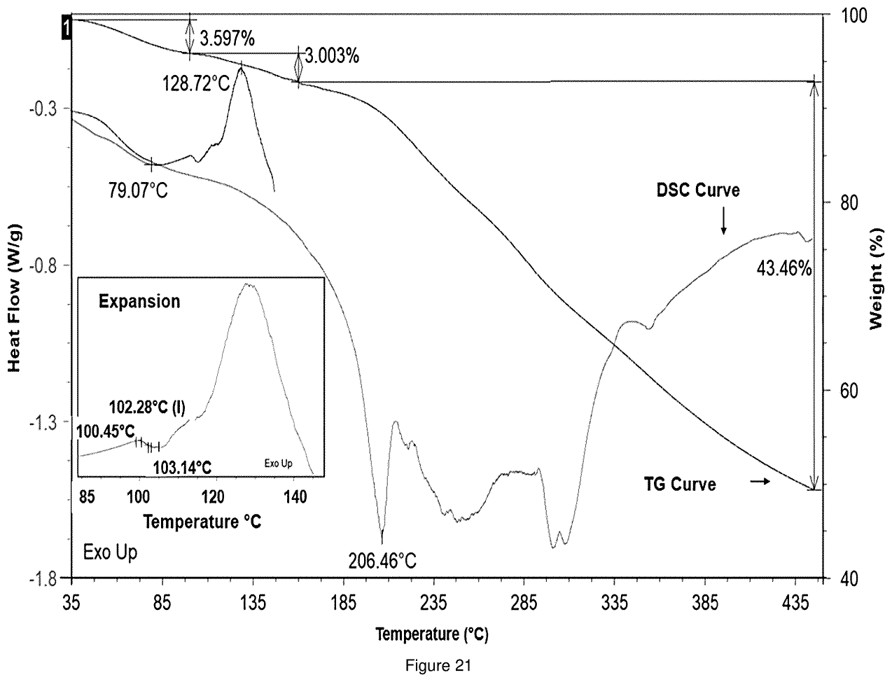

FIG. 21 shows the thermal analysis of the solid obtained by rapid evaporation of ethanol/water. In the DSC thermogram an endothermic event at 79.07° C. is observed in the first heating (red curve), followed by a glass transition at 102° C. and finally a crystallization at 128° C. TGA analysis reveals mass loss at 80° C. which coincides with the exothermic event in the DSC, followed by another loss at approximately 130° C. which coincides with the exothermic process of the DSC. In total, these losses add up to 6.6% and can be attributed to two processes of desolvation of water and ethanol molecules (cal. 6.93%). After the glass transition, the solvent interactions are broken, the solvent begins to evaporate and the residue begins to crystallize (exothermic event in the DSC at 128° C.). This crystallization process seems to be irreversible, because in the second heating (green curve) endothermic or exothermic events below 206° C., the temperature at which the solid begins to sublimate, are not appreciated. The presence of several sublimation points could be attributed to the fact that the co-amorphous solid separates with heating, after the glass transition event. Based on the above, it can be concluded that the solid GTN:ARG 1:4 obtained by rapid evaporation of ethanol is a co-amorphous solvate with stoichiometry GTN:ARG:ethanol 1:4:1.

### Example 12. Comparison of the New Co-Amorphous Solid Form GTN:ARG 1:4 Obtained by Rapid Evaporation in Ethanol/Water, Grinding in Water, Rapid Evaporation in Water and Slurry in Water

### X-Ray Powder Diffraction of GTN:ARG 1:4 NSF

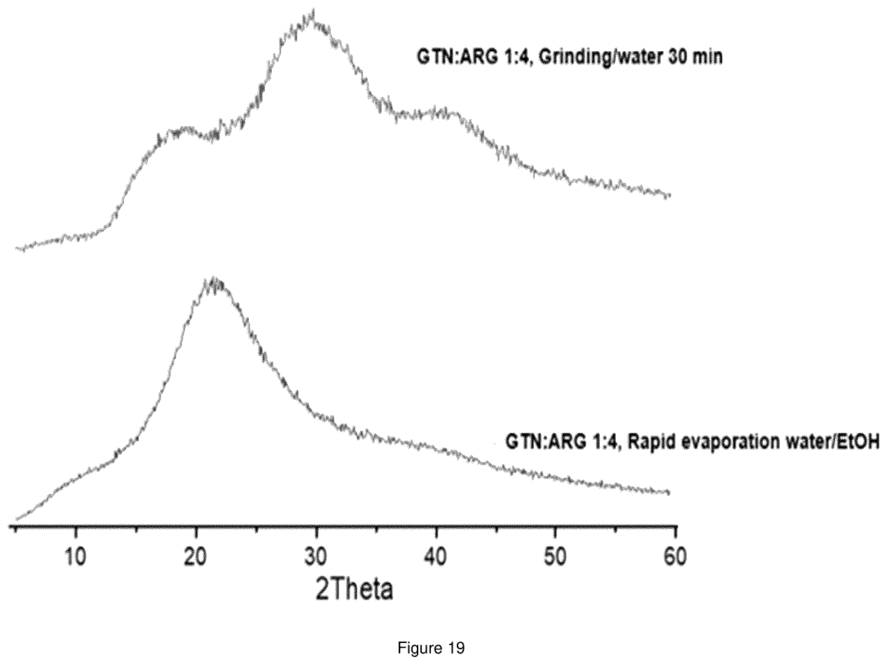

FIG. 19 shows diffractograms of the solid obtained by rapid evaporation of water/ethanol and the one obtained by wet grinding of Example 8. The diffractogram for rapid evaporation in ethanol/water has a single diffraction band without diffraction peaks compared to the diffractogram of the solid obtained by grinding in water.

### FT-IR Spectroscopy of GTN:ARG 1:4 NSF

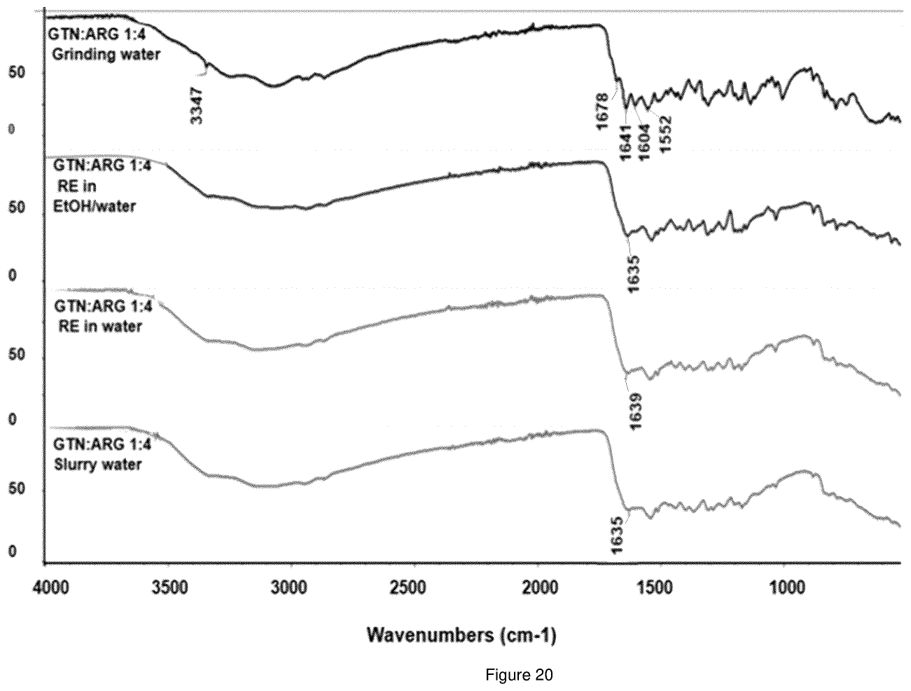

FIG. 20 presents the spectra of GTN:ARG 1:4 solids obtained by wet grinding and rapid evaporation (RE) in ethanol/water, as well as the GTN:ARG 1:4 semisolids obtained from rapid evaporation (RE) in water and slurry in water.

The pattern of vibrational bands of the spectrum of the solid GTN:ARG 1:4 obtained by rapid evaporation in ethanol/water shows a widening and loss of the definition of the vibrational bands, unlike the product of obtained by grinding in water where the bands appear more defined. The band assigned to the vibration of group —NH₂ of arginine observed at 3347 cm⁻¹ in the spectrum of the solid obtained by grinding in water, disappears in the spectrum corresponding to the solid obtained by rapid evaporation of ethanol/water, while the bands at 1678, 1641 and 1604 cm 1 corresponding to the vibrations of the NH₂ bonds, C—NH₂ and C—N of arginine, and C═C of genistein are joined together in a single broad band at 1635 cm-1.

### Example 13a. New Co-Amorphous Solid Forms of Naringenin:Arginine (NRG:ARG)

The preparation of the NRG:ARG system was started using the technique of rapid evaporation of the solvent. A mixture NRG:ARG 1:1 was added to approximately 25 ml of water. However, despite leaving it in rotation and in a water bath at 80° C., the mixture was not dissolved. Thus, the stoichiometry was increased to a NRG:ARG 1:2 ratio, which was not dissolved either. Finally, the stoichiometry NRG:ARG 1:3 presented a rapid and complete dissolution of the mixture. The pressure was decreases until complete evaporation of the solution, obtaining a completely dry and manageable solid.

### Physical Analysis of NRG:ARG 1:3 NSF

The obtained solid exhibited an intense yellow color.

### X-Ray Powder Diffraction of NRG:ARG 1:3 NSF

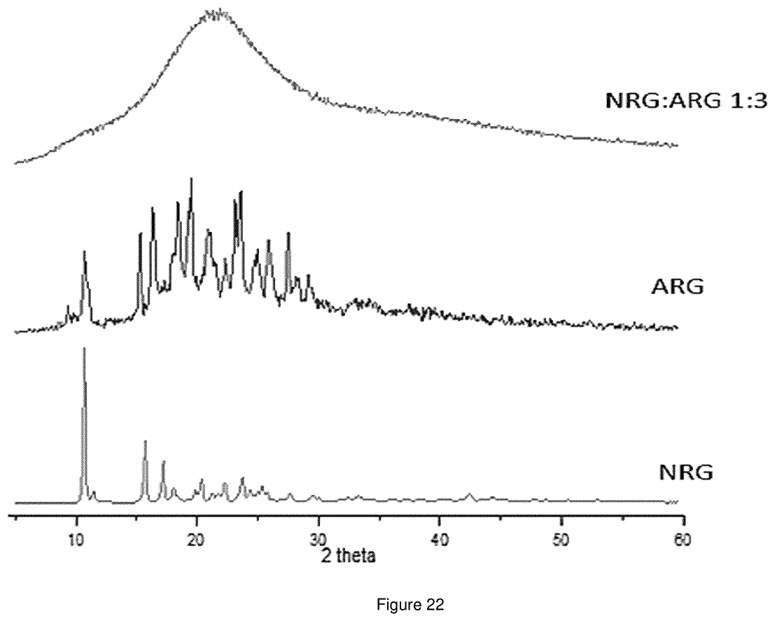

X-ray powder diffraction was performed for the raw materials and the solid NRG:ARG 1:3. Diffractograms are shown in FIG. 22. A diffuse halo characteristic of amorphous solids is observed. The absence of diffraction peaks indicates that the solid is completely amorphous and there are no crystalline residues of the raw materials.

### FT-IR Spectroscopy of NRG:ARG 1:3 NSF

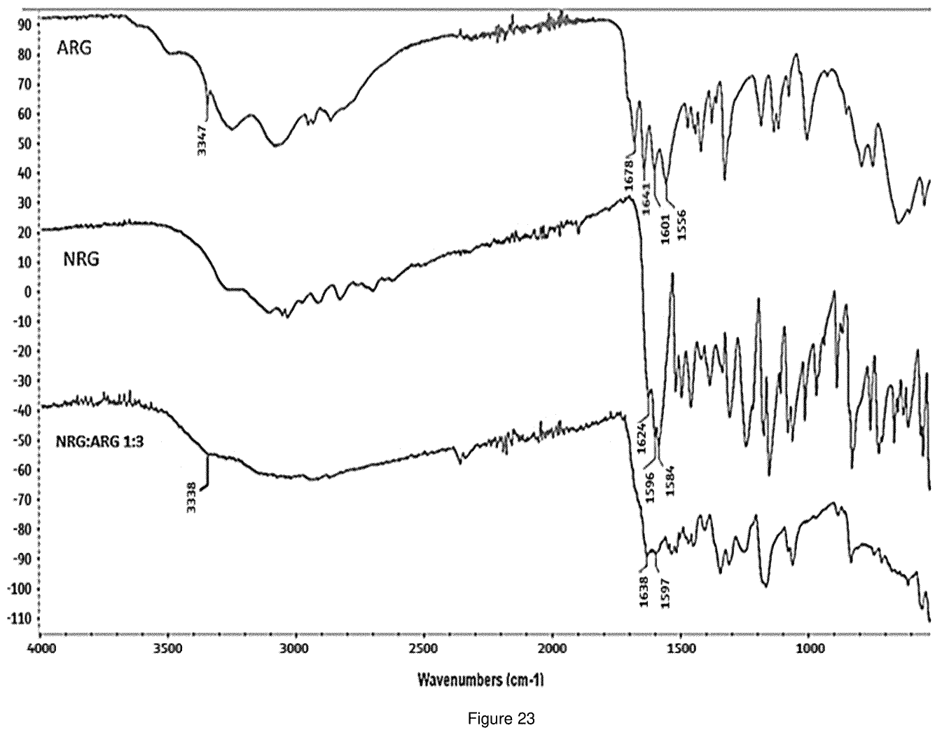

FIG. 23 shows the FT-IR spectrum obtained for this NSF and the raw materials. The spectrum of NRG:ARG 1:3 shows a wide and poorly defined band pattern that confirms the amorphous nature of the solid. Likewise, it can be noted that the band observed at 3344 cm⁻¹ in the spectrum of arginine, which was assigned to the stretching vibrations of the NH₂ bonds, shifts and joins in a small and wide band with a maximum of 3338 cm⁻¹ on the spectrum of NRG:ARG 1:3. In addition, the bands observed at 1678, 1641 and 1601 cm⁻¹ corresponding to the vibrations of the NH₂, C—NH₂ and C—N bonds of arginine, and the bands at 1624 and 1598 cm⁻¹ assigned to the C═O and C═C vibrations of naringenin, are joined in two wide and poorly defined bands at 1638 and 1597 cm⁻¹. Based on this observation, it can be concluded that NRG establishes intermolecular interactions with arginine.

### NSF Thermal Analysis of NRG:ARG 1:3 NSF

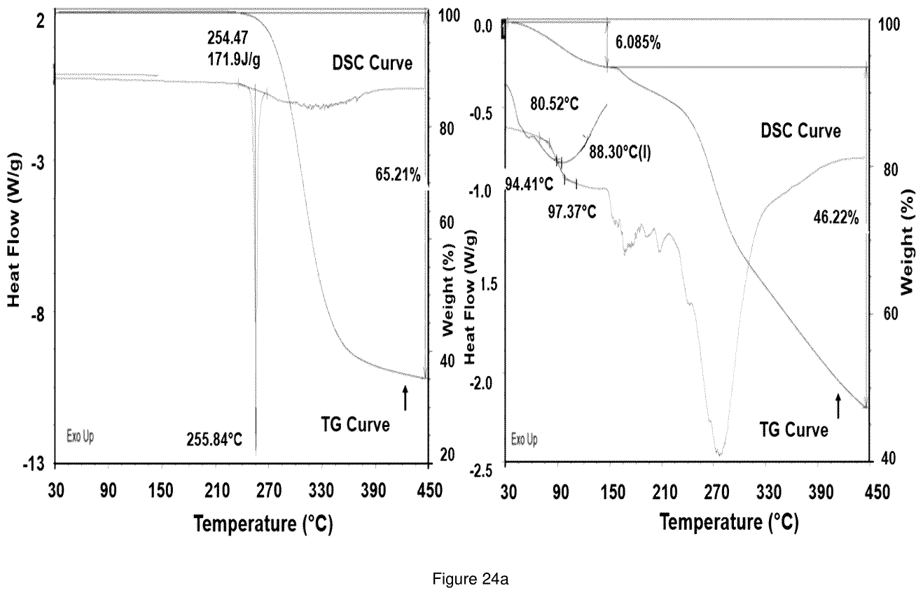

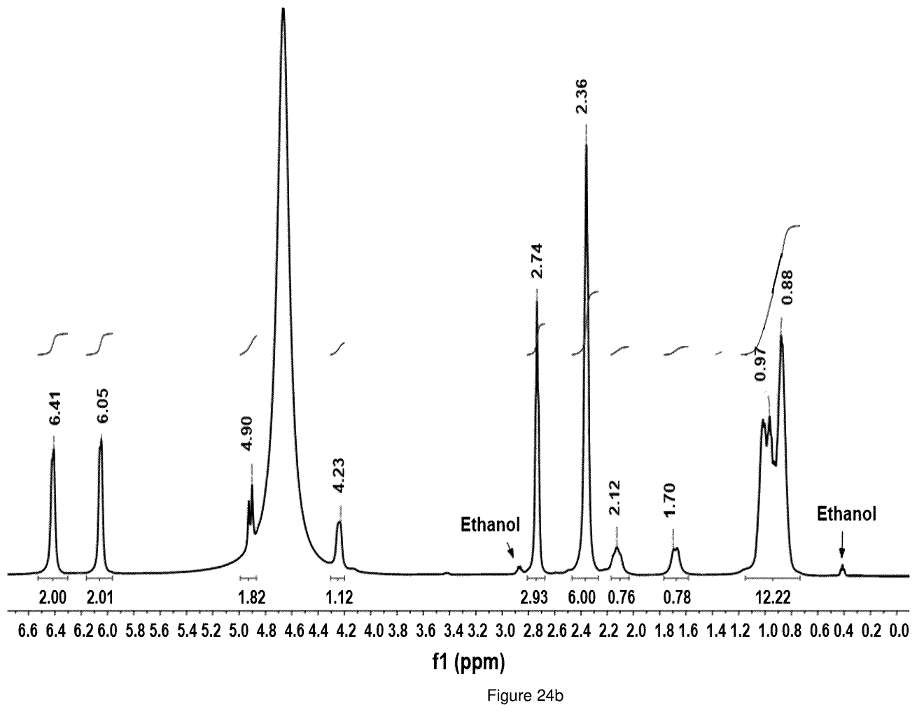

FIG. 24a shows the thermograms of naringenin (left) and NRG:ARG 1:3 NSF (right). For naringenin, the DSC thermogram only shows one endothermic event at 255° C. and a mass loss of 65% that was assigned to a process of fusion/decomposition of naringenin. On the contrary, the thermogram of DSC and TGA for NRG:ARG 1:3 in the first heating performed, shows an endothermic event at 94° C. that, due to the loss of mass of 6.08% in the TGA, was assigned to a process of dehydration of the co-amorphous solid, losing 3 molecules of water (calc. 6.36%). In the second heating a single glass transition with a midpoint at 88° C. is apparently observed, which confirms that pure co-amorphous systems were obtained instead of amorphous physical mixtures. Subsequently, processes of fusion/decomposition of the co-amorphous solid appear in the thermogram.

### Solubility Tests of NRG:ARG 1:3 NSF

The reported solubility of naringenin is 4.38 μg/mL, whereas the solubility of the naringenin complex with hydroxypropyl-6-cyclodextrin is 1.2761 mg/mL. (see Wen et al., Molecules 2010, 15, 4401-4407).

Supersaturated solutions of naringenin and NRG:ARG 1:3 NSF were prepared from 0.5 ml of water. In the case of naringenin, 22 mg was added and practically it did not dissolve at all. Surprisingly, for NRG:ARG 1:3 NSF, 268.2 mg was added to the same volume, allowing complete dissolution. This is equivalent to a solubility of 536.4 mg/mL.

### Example 13b. New Co-Amorphous Solid Solvate of NRG:ARG:Ethanol 1:3:1 Obtained by Rapid Evaporation of Ethanol/Water

Arginine was initially dissolved in a round-bottom flask with a minimum amount of water. Naringenin and 30 mL of ethanol were subsequently added to the flask, until the mixture was completely dissolved, and was rotavaporated to dryness. The obtained solid was analyzed by NMR¹H. The NMR¹H spectrum in D₂O of FIG. 24b shows two signals 6.05 and 6.41 ppm that were assigned to the hydrogens of the phenolic ring of NRG, each one integrating for 2 hydrogens. A doublet is observed at 4.9 ppm, which integrates for 1 hydrogen (the integral corresponds to 1.8, but the signal overlaps with the water signal) and another peak at 4.23 ppm also integrating for 1 hydrogen were assigned to the hydrogen that lies between the aromatic OH group and the pyranone ring, and the hydrogen from the pyranone ring of NRG. For arginine, hydrogen signals are observed from alpha carbon to carboxyl at 2.74 ppm and integrating for 3 hydrogens. At 2.36 ppm, a signal integrating for 6 hydrogens was assigned to the —CH₂ bond to the guanidinium group. Finally, a multiple integrating signal for 12 hydrogens was assigned to the methylene hydrogens of the arginine chain. It is worth mentioning that a triplet and a multiplet are observed at 0.41 and 2.8 ppm respectively, which are assigned to an ethanol molecule. Based on this, it can be concluded that the naringenin coamorphous obtained has a stoichiometry of NRG:ARG:ethanol of 1:3:1.

### Example 14. New Co-Amorphous Solid Forms of Apigenin:Arginine (APG:ARG) 1:4 Obtained by Dry Slurry, Slurry in THF and Slurry in Ether

Dry slurry, slurry in THF and ether were performed for APG:ARG 1:4. The obtained products were characterized by X-ray powder diffraction and FT-IR spectroscopy.

### X-Ray Powder Diffraction of Apigenin-Arginine Slurry

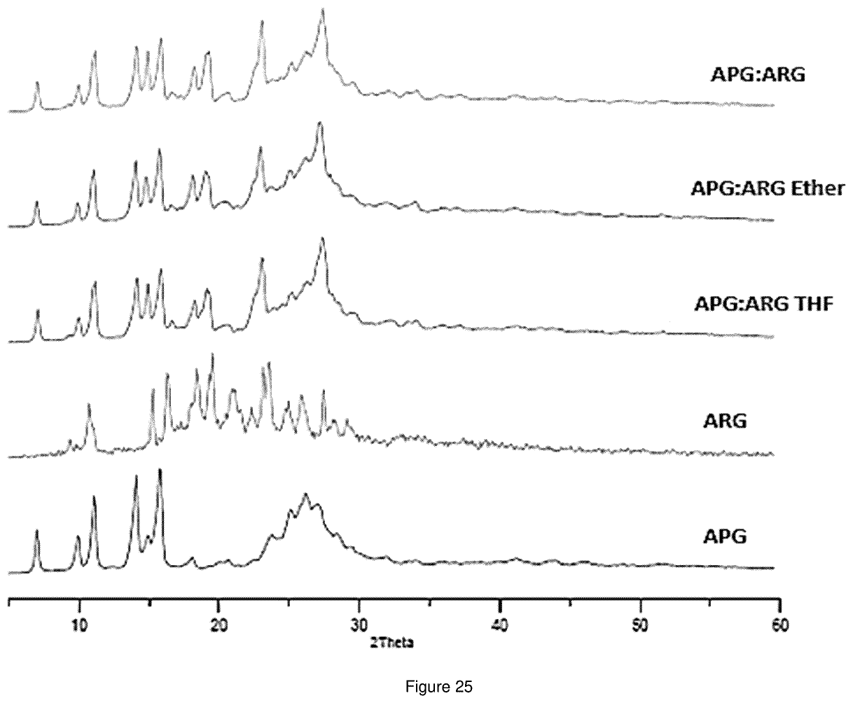

FIG. 25 shows the X-ray powder diffractograms of the solids obtained by dry slurry, slurry in THF and slurry in ether, comparing them with the raw materials. It can be observed that in all three cases the diffractograms obtained from the slurries coincide with the sum of the diffractograms of the raw materials, concluding that there is no intermolecular interaction between apigenin and arginine, thus resulting in a physical mixture.

### FT-IR Spectroscopy of Apigenin-Arginine Slurry

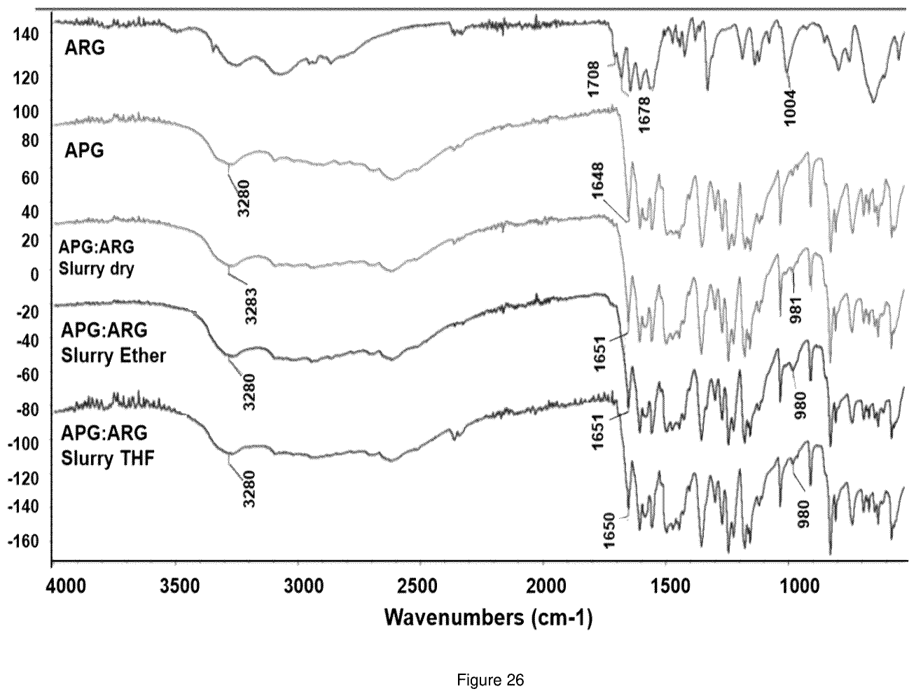

FIG. 26 shows the FT-IR spectra of raw materials arginine (ARG) and apigenin (APG), dry slurry, slurry in ether and slurry in THF. The pattern of absorption bands in these slurries is similar to that of apigenin, where the shifts observed for the bands of the hydroxyl and carbonyl groups of apigenin, are not very large, being found between 3280 to 3283 cm⁻¹ and 1648 and 1651 cm⁻¹ respectively. This indicates that apigenin does not establish intermolecular interactions with arginine. On the other hand, the bands assigned to the stretching of the carbonyl group of carboxylic acid in arginine at 1708 and 1678 cm⁻¹, disappear completely in the spectra of slurries, possibly due to changes related to the establishment of interactions of arginine with the solvent. The presence of arginine can be observed in the spectra of slurries as a slight band between 980 to 981 cm⁻¹, which corresponds to the intense band at 1004 cm⁻¹ in the arginine spectrum.

### Example 15. New Co-Amorphous Solid Form of Apigenin:Arginine (APG:ARG) 1:8 Obtained by Rapid Evaporation in Water and DMSO

An APG:ARG 1:8 NSF was prepared using the technique of rapid evaporation of water and DMSO. A non-dry thick solid was obtained, therefore it was not possible to obtain the X-ray powder diffractogram.

### FT-IR Spectroscopy of Apigenin-Arginine by Rapid Evaporation of Water and DMSO

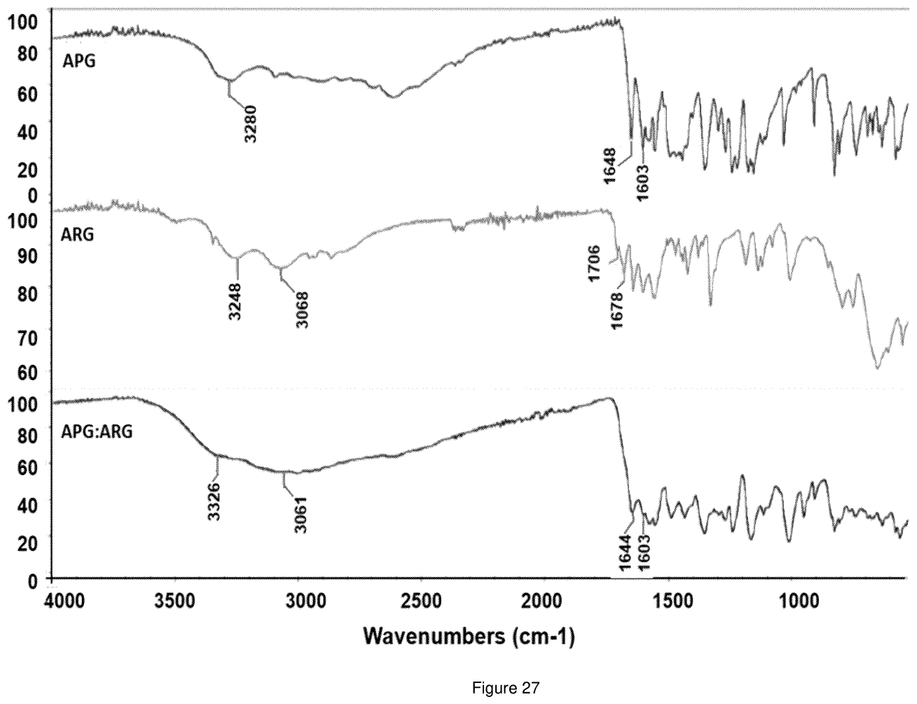

FIG. 27 shows the FT-IR spectra of the raw materials and the solid obtained. A noticeable change in the pattern of bands in the region from 3500 to 2500 cm⁻¹ is observed. The band at 3280 cm⁻¹ assigned to the vibrations of the O—H bond the hydroxyl groups of apigenin undergoes a significant shift to 3326 cm⁻¹ in the slurry. Similarly, the bands at 3248 cm⁻¹ and 3068 cm⁻¹ assigned to the stretching of O—H and N—H bonds in the arginine spectrum are widened and joined in a single band with a maximum of 3061 cm⁻¹ in the spectrum of the product obtained from rapid evaporation with water and DMSO. The most obvious changes are observed in the region of the carbonyls of apigenin and arginine at 1648 cm⁻¹ and 1706 cm⁻¹ and 1678 cm⁻¹ respectively, which join in a single relatively wide band to 1644 cm⁻¹ in the solid obtained. These shifts are attributed to the establishment of intermolecular interactions between apigenin and arginine. It is worth mentioning that the band assigned to aromatic C═C stretching in apigenin (1603 cm⁻¹) does not show any shift in the NSF. This indicates that the aromatic part of apigenin does not interact with arginine. The loss in the definition of vibration bands is characteristic of amorphous systems, although it could be attributed to the presence of solvent or a hydration process of the sample.

### Example 16. New Co-Amorphous Solid Forms of Apigenin:Arginine (APG:ARG) 1:8 Obtained by Rapid Evaporation in Water and Dry Grinding

Solids were also prepared by rapid evaporation in water and by dry grinding using a 1:8 stoichiometry. The two obtained samples were completely dry powders and easily manipulated. However, the sample obtained by rapid evaporation of water has a more intense and lumpy yellow color than the solid obtained by dry grinding.

### X-Ray Powder Diffraction of the Solids APG:ARG Obtained by Rapid Evaporation in Water and by Dry Grinding

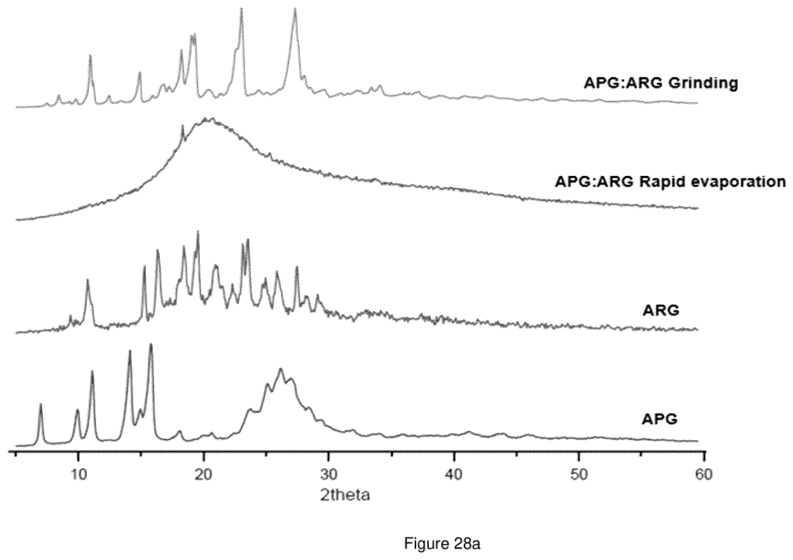

FIG. 28a shows the powder diffractogram of the APG:ARG 1:8 solid obtained by solvent rapid evaporation and that obtained by dry grinding, as well as the raw materials. The spectrum corresponding to rapid evaporation shows a diffuse halo characteristic of an amorphous solid, however, it is also possible to identify small diffraction peaks at 18 and 25 2theta that could be attributed to a small amount of residual arginine that probably did not dissolve completely. For dry grinding technique, the sample has diffraction peaks that correspond to arginine.

FIG. 28b shows the diffractogram of the APG:ARG 1:8 solid obtained by dry grinding and compares it with that of the physical mixture of apigenin-arginine previously obtained. It is noted that the diffraction peaks of arginine remain unchanged. However, the diffractogram of the solid obtained by dry grinding does not reflect the diffraction peaks that appear at 7, 14 2theta in the diffractogram of the physical mixture. In addition, the solid obtained by dry grinding has other small diffraction peaks. This change could be due possibly to obtaining a solvate of apigenin.

### FT-IR Spectroscopy Apigenin-Arginine 1:8 Obtained by Rapid Evaporation of Water and by Dry Grinding

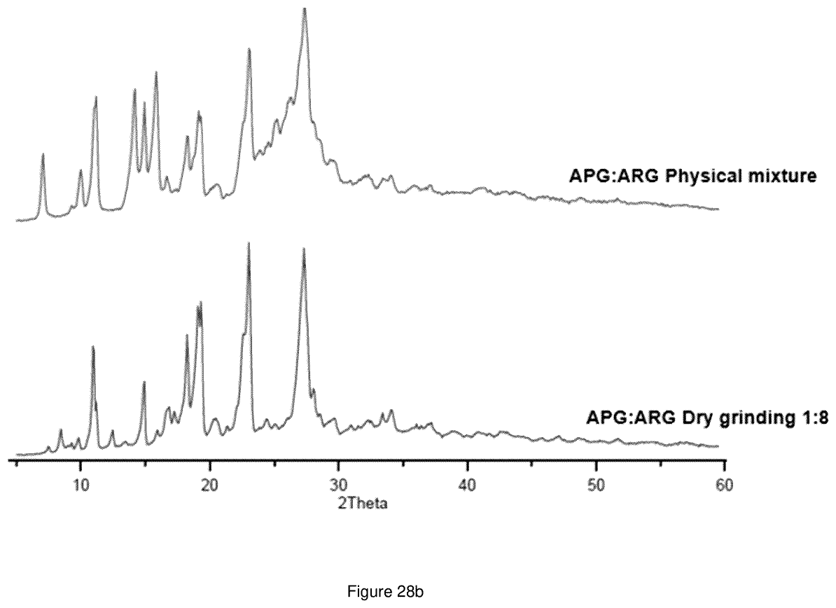

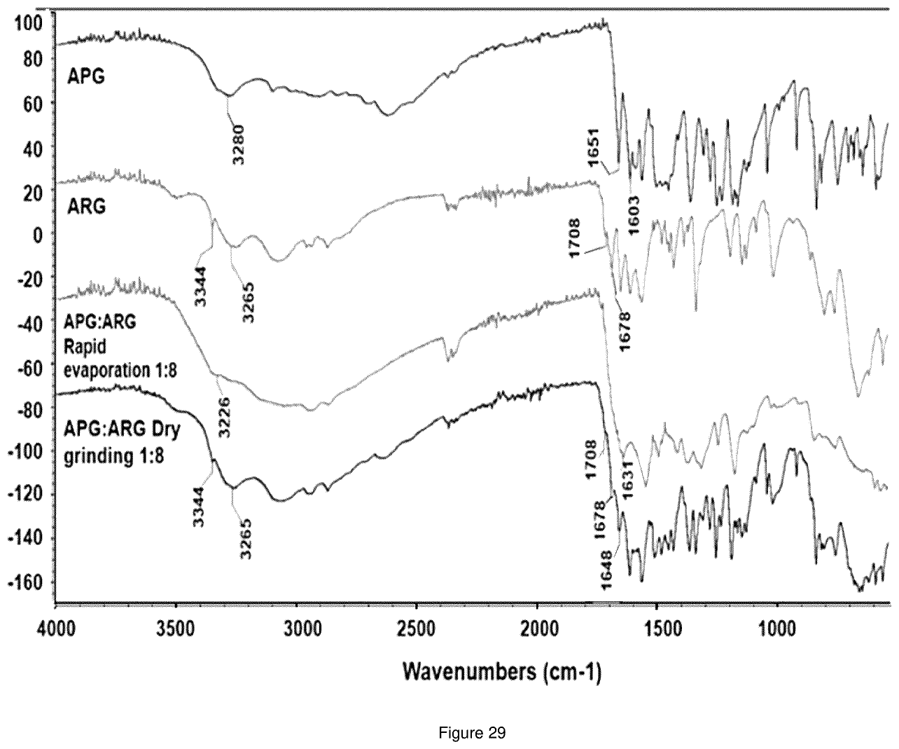

FT-IR spectroscopy was used to determine the possible establishment of intermolecular interactions between apigenin and arginine in APG:ARG 1:8 solids, as shown in FIG. 29. For the sample APG:ARG 1:8 obtained by rapid evaporation of water, there is a widening and an important loss of definition of the vibration bands that is attributed to the process of amorphization of the obtained solid. A noticeable change in the pattern of bands is also observed in the region from 3500 to 2500 cm⁻¹. The band at 3280 cm⁻¹ assigned to the vibrations of the O—H bonds of the hydroxyl groups of apigenin, and the bands at 3244 cm⁻¹ and 3265 cm⁻¹ assigned to the stretching vibrations of the O—H and N—H bonds in the arginine spectrum, widen and join in a single band with a maximum of 3326 cm⁻¹. The most obvious changes are observed in the region of apigenin and arginine carbonyls at 1651 cm⁻¹ and 1708 cm⁻¹ and 1678 cm⁻¹ respectively, which join in a single broad band at 1631 cm⁻¹. The band assigned to the aromatic C═C stretch in apigenin (1603 cm⁻¹) and the vibrational band at the same displacement in the arginine spectrum, disappear completely. Thus, it is concluded that apigenin and arginine interact in the solid state through the hydroxyl, carbonyl and aromatic groups that exist in the two components. In addition, the loss of definition and widening of the vibrational bands indicates that the obtained phase could be a co-amorphous solid.

As for the solid with stoichiometry 1:8 obtained by dry grinding, the bands in the region of O—H and N—H vibrations (3500 to 3000 cm⁻¹) of apigenin and arginine do not suffer any shift, resulting in the sum of the vibrational bands of apigenin and arginine. Similarly, the bands corresponding to the carbonyl groups of arginine are observed identical and the shifts remain unchanged. It is concluded that apigenin and arginine do not form intermolecular interactions in this solid form.

### Thermal Analysis of the NSF APG:ARg 1:8 Obtained by Rapid Evaporation of Water

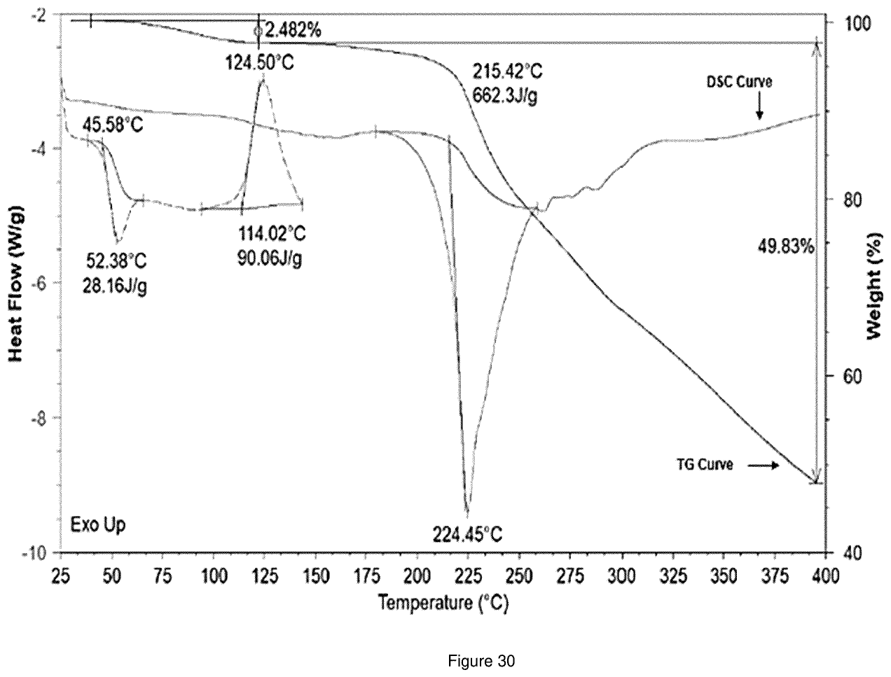

Once it was determined that the APG:ARG 1:8 solid obtained by rapid evaporation of water corresponds to a co-amorphous solid, the thermal analysis was performed. The results are shown in FIG. 30. Initially the sample was heated to 150° C., then cooled to 30° C. and reheated. In the respective thermogram of the first heating (in dashed lines) an endothermic event is clearly observed at 45° C. The mass loss of 2.48% observed in the TGA seems to be a dehydration process. Since this co-amorphous system was elaborated through a process of rapid evaporation in water, it corresponds to the loss of one mole of water (cal. 2.22%). Continuing with the heating, an exothermic event can be observed without loss of mass, which corresponds to a crystallization of the components. In the second heating, there is no thermal event until 215° C., where the decomposition of the sea is observed as a well-defined endothermic event and with a weight loss of almost 50% of as observed in the TGA. Based on the above, it can be deduced that both the desolvation and crystallization are irreversible processes. That is why in the second heating, these events do not occur, and only the decomposition of the mixture is observed. Based on the above it can be concluded that the obtained API:ARG 1:8 NSF is a co-amorphous hydrate.

### Example 17. Trials with Diosmin (DIS) and Arginine (ARG)

Dry slurry and slurries in THF and ether were prepared and characterized by X-ray powder diffraction and FT-IR spectroscopy.

### X-Ray Powder Diffraction of Diosmin-Arginine Slurries

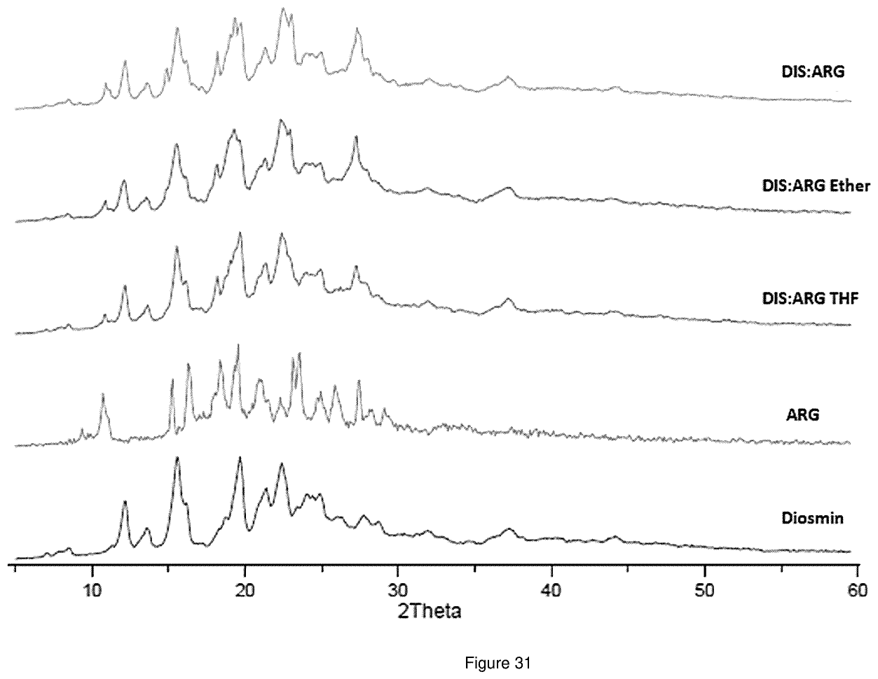

The diffractograms of the slurries were compared with the diffractograms of the raw materials, as shown in FIG. 31. In the three cases the diffractogram obtained from the slurries coincides with the sum of the diffractograms of the raw materials, concluding that there is no intermolecular interaction between diosmin and arginine, and the samples correspond to physical mixtures.

### FT-IR Spectroscopy of Diosmin-Arginine Slurry

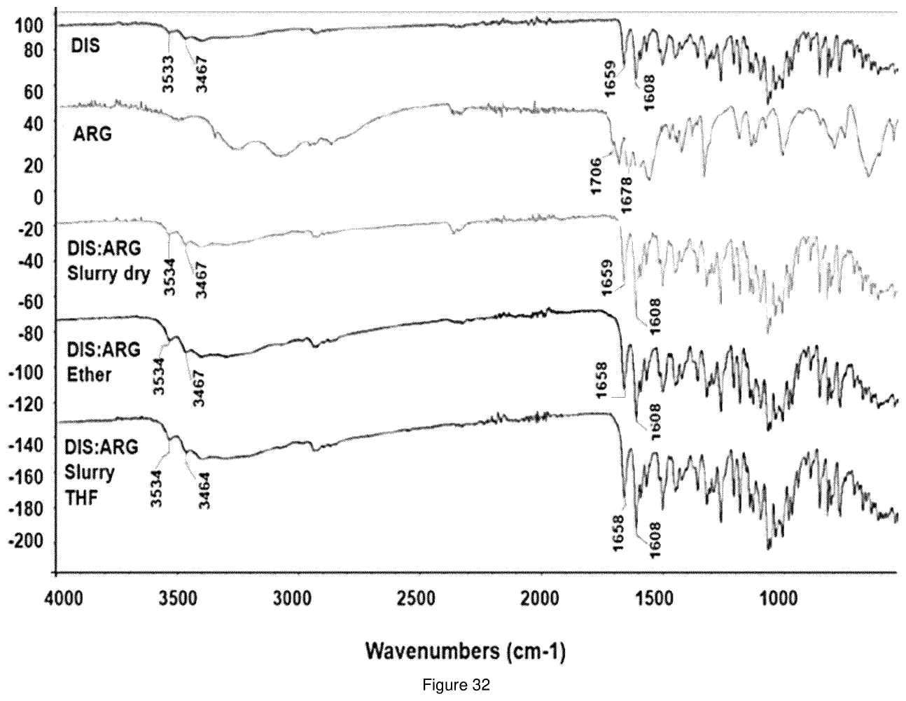

FIG. 32 shows the FT-IR spectra of the raw materials arginine (ARG) and diosmin (DIS), the dry slurry and slurries in ether and THF. The pattern of the slurries is similar to that of the diosmin, where the shift of the bands of the hydroxyl groups at 3533 and 3467 cm⁻¹ in the spectrum of the diosmin, appear at 3534 cm⁻¹ and 3467-3464 cm⁻¹ respectively. Similarly, the bands assigned to the carbonyl and aromatic group of diosmin at 1659 cm⁻¹ and 1678 cm⁻¹ respectively, show only shifts of 1 cm⁻¹ with respect to the slurries, concluding that diosmin does not establish new interactions with arginine or solvent. On the other hand, the bands assigned to the carbonyl stretching vibration of the carboxylic acid in arginine observed at 1708 and 1678 cm⁻¹, completely disappear in the spectra of the slurries. This is possibly due to changes related to the establishment of interactions of arginine with the solvent. Based on the above, it can be concluded that the products obtained from slurries are only physical mixtures.

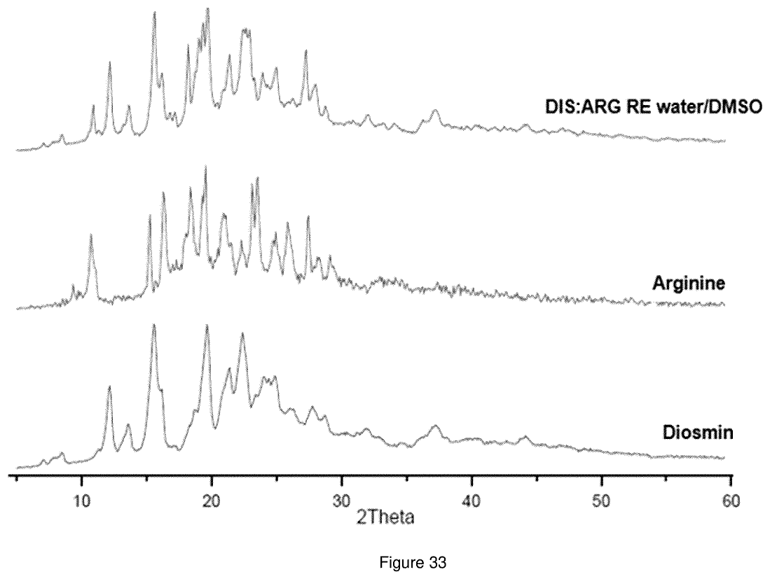

A solid was also obtained using the technique of rapid evaporation with water and DMSO, with diosmin and arginine. This sample turned out to be a powder that was characterized by X-ray diffraction. FIG. 33 shows the diffractograms of the raw materials and the obtained solid. The diffractogram of the solid obtained by this technique coincides with the sum of the diffractograms of the raw materials, concluding that it is a physical mixture.

### Example 18. New Co-Amorphous Solid Form of Rutin:Lysine (RTN:LYS) 1:3

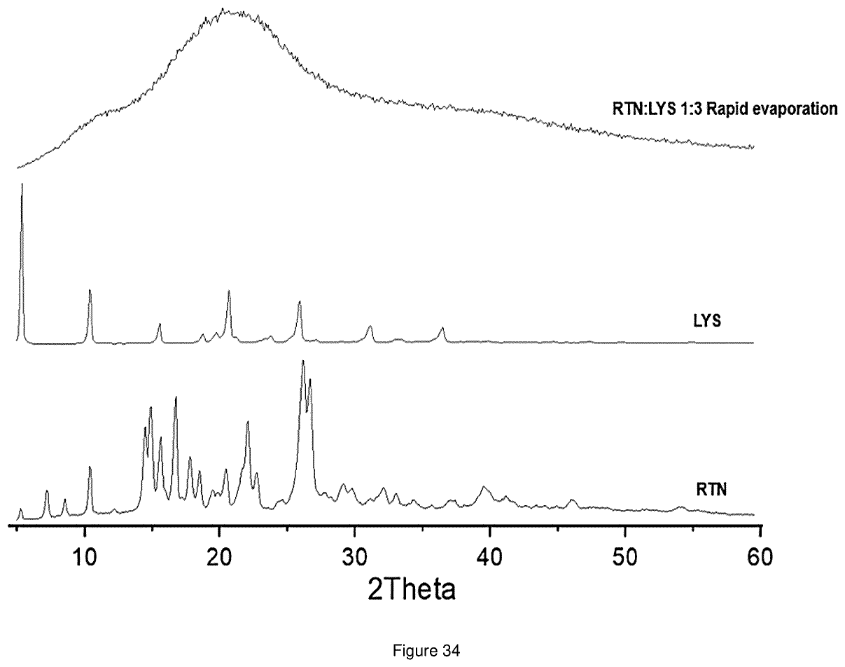

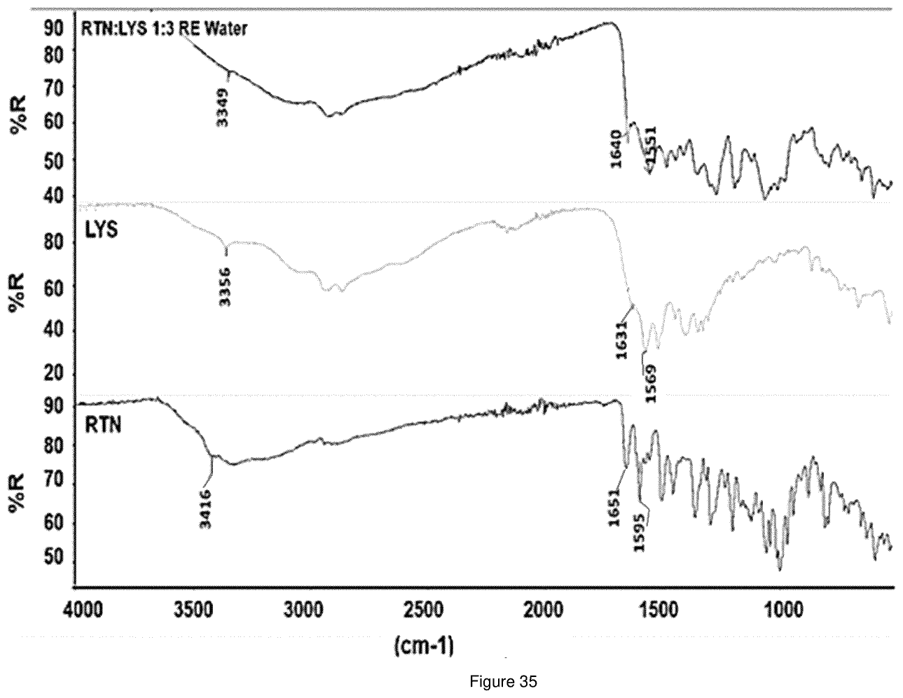

An NSF of rutin and lysine was prepared by rapid evaporation (RE) from water using a 1:3 stoichiometry. FIG. 34 shows the X-ray powder diffractograms of the raw material (lysine and rutin) and the RTN:LYS 1:3 NSF. FIG. 35 shows the FT-IR spectra of the raw materials lysine (LYS) and rutin (RTN), and the RTN:LYS 1:3 NSF.

### Example 19. New Co-Amorphous Solid Form of Naringenin:Lysine (NRG:LYS) 1:3

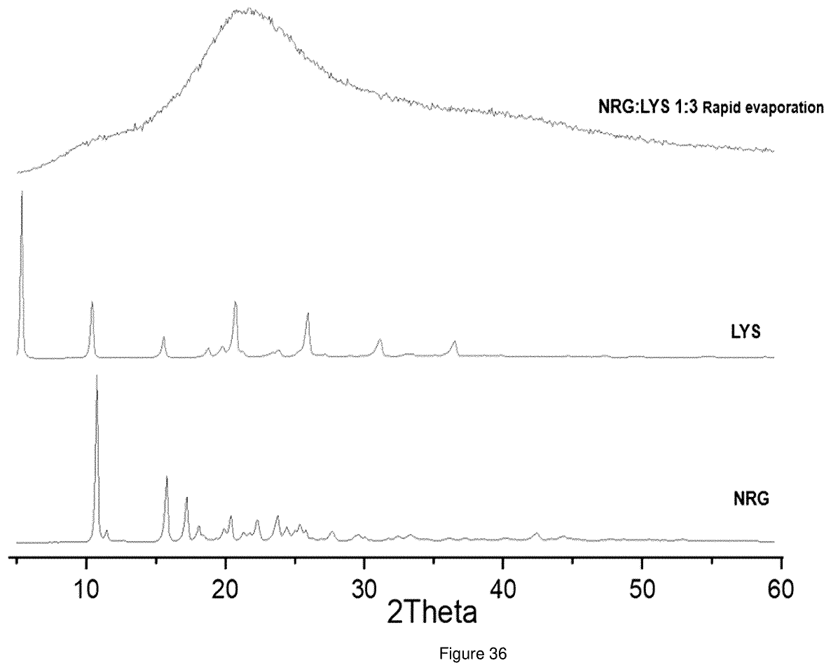

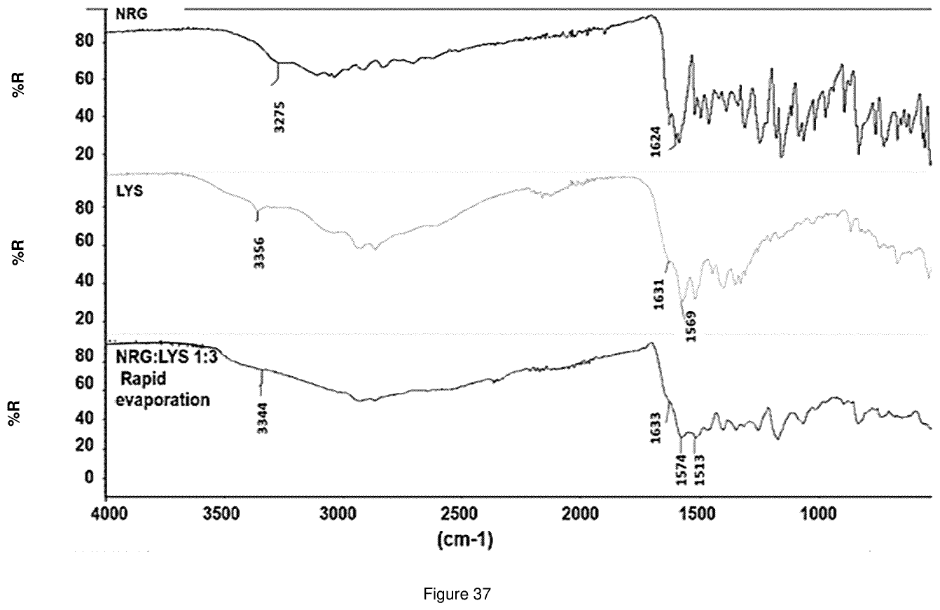

An NSF of naringenin and lysine with a 1:3 stoichiometry was prepared by rapid evaporation from water. FIG. 36 shows the X-ray diffractograms of raw material powders lysine/LYS) and naringenin (NRG), and the NRG:LYS 1:3 NSF. FIG. 37 shows the FT-IR spectra of raw materials (lysine and naringenin) and the NRG:LYS 1:3 NSF.

### Example 20. New Co-Amorphous Solid Form of Apigenin:Arginine (APG:ARG) 1:3 Obtained by Rapid Evaporation in Water and Ethanol

### X-Ray Powder Diffraction of the Solid APG:ARG 1:3 Obtained by Rapid Evaporation in Water and Ethanol

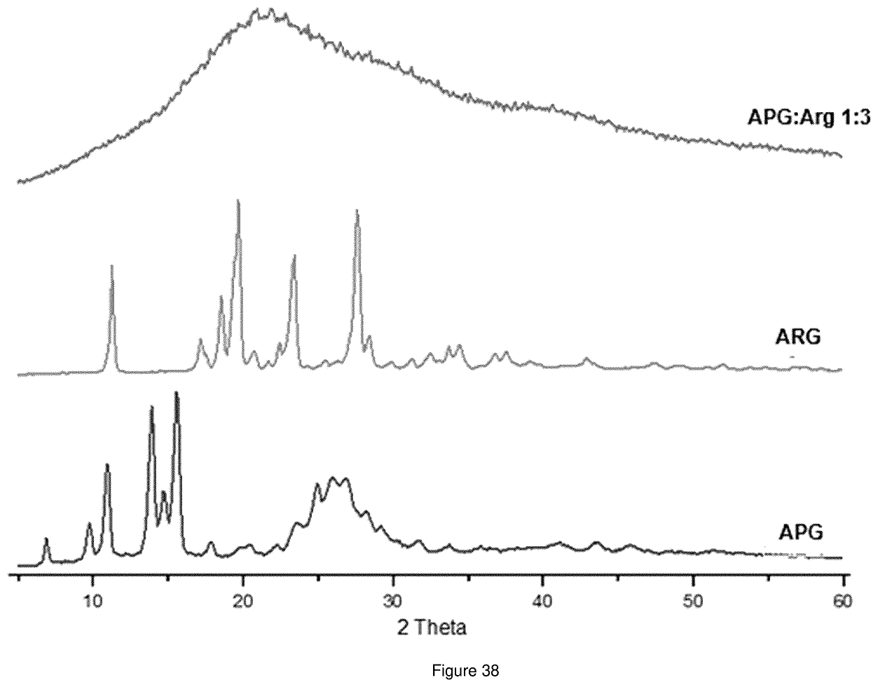

The coamorphous NSF of API:ARG 1:3 was obtained by rapid evaporation of solvent, by dissolving 150 mg a stoichiometric mixture 1:1 of apigenin and arginine using 20 ml of a mixture of ethanol with 1 ml of water. The mixture was left in rotation in a water bath at 80° C. for a period of 10 minutes. However, the mixture was not completely dissolved. Thus, one additional equivalent of arginine was added, to obtain a stoichiometry of 1:2 and it was left in rotation in water bath for other 20 minutes. Once again, a slight precipitate was observed, corresponding to apigenin. Therefore, another mole of arginine was added to obtain a stoichiometric ratio of 1:3. In this case, the residual apigenin was completely solubilized. Once the apigenin was dissolved, evaporation of the solvent under vacuum was carried out. The sample was evaporated to dryness giving an intense yellow solid which was scraped off and characterized by X-ray powder diffraction. The diffractogram obtained was compared with the diffractograms of the raw materials APG and ARG as shown in FIG. 38.

As can be seen from FIG. 38, the diffractograms of the raw materials show a pattern of well-defined diffraction peaks, indicating their crystallinity. The X-ray diffractogram of the NSF obtained by rapid evaporation shows a diffuse diffraction halo with a maximum in about 2 Theta=20, and no diffraction peaks, showing that the solid is amorphous.

### FT-IR Spectroscopy of APG:ARG 1:3 Obtained by Rapid Evaporation in Water and Ethanol

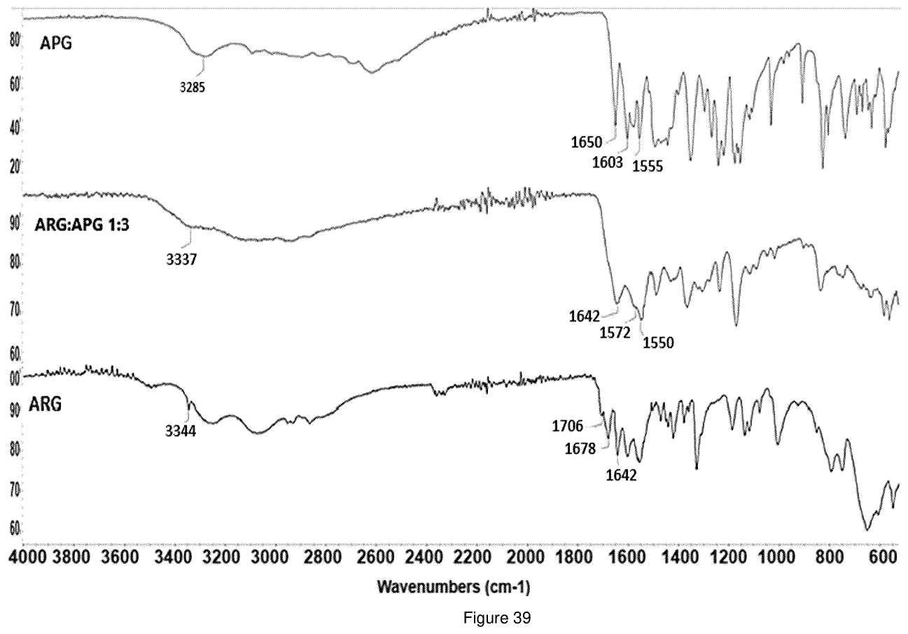

To determine the presence of intermolecular interactions between APG and ARG, the raw materials and solid obtained by rapid evaporation were characterized by IR spectroscopy (see FIG. 39). The spectrum of APG shows a broad absorption band at 3285 cm⁻¹ due to the C—H vibrations of of the aromatic rings. At 1651 cm⁻¹ the characteristic band of the C═O stretch of the carbonyl group is observed. At 1603 and 1555 cm⁻¹ two bands corresponding to the C═C stretches of the aromatic groups are observed. For ARG, the most important bands for arginine are a small, well-defined band at 3344 cm⁻¹ that was assigned to the stretching of the NH₂ group at 1706 cm⁻¹, a small shoulder assigned to the C═O stretch of the carboxyl group, and two more bands at 1678 and 1641 cm⁻¹ assigned to the asymmetric bending vibrations of the NH₂ group and the N—H bending vibration of the guanidinium ion, confirming that ARG is found in zwitterionic form. The IR spectrum for APG:ARG 1:3 shows fully widened vibrational bands confirming the amorphicity of these mixtures. The bands observed at 3285 cm⁻¹ in APG and the well-defined band at 3344 cm⁻¹ in ARG disappear and join together in a single broad band between 3431 and 3200 cm⁻¹, indicating that the hydroxyl and amino groups form new intermolecular interactions. Likewise, the vibrational bands of the carbonyl groups and aromatic carbons at 1603 and 1555 cm 1 in APG, and the bands assigned to the carbonyl, guanidinium and amino group of ARG (1706, 1678 and 1642 cm⁻¹ respectively), join into two wide bands between 164 and 1572 cm⁻¹ in APG:ARG 1:3, indicating that these functional groups establish new molecular interactions.

### Complexes in Dissolution of the Obtained NSF

In the case of the co-amorphous compounds obtained, it is important to note that all of them remain as stable complexes even when they are in solution.

### Preclinical Study of the Flavonoid NSF

The beneficial effect of flavonoid NSF in metabolic syndrome and intestinal microbiota was evaluated in a preclinical study. The flavonoid NSF was selected from Genistein:Arginine 1:4, Fisetin:Arginine 1:3, Quercetin:Arginine 1:3, Rutin:Arginine 1:3 and Naringenin:Arginine 1:3, among others.

Flavonoid NSF were administered orally to C57BL6 mice fed with a high-fat and high-sucrose diet (HFSD) or a normal diet, for a period of 12 to 14 weeks. Glucose tolerance and insulin resistance tests were performed. Glucose, insulin, body weight, food intake, triglycerides, cholesterol, leptin, adiponectin, IL-6, and C-reactive protein levels, among others, were evaluated. In addition, a change in the microbiota was determined after the administration of HFSD diet and NSF flavonoids.

Fifty-six (56) male mice of the C57BL/6 strain, between 7 and 9 weeks old, were used in the study. The microbiota of the mice was unified during 4 weeks. The mice were randomly assigned to seven experimental groups:
Group 1. Control Diet or normal diet AIN-93 (American Institute of Nutrition 93)
Group 2. HFSD: High fat and sucrose diet
Group 3. HFSD+NSF Genistein:Arginine:ethanol 1:4:1
Group 4. HFSD+NSF Fisetin:Arginine:ethanol 1:3:1
Group 5. HFSD+NSF Quercetin:Arginine 1:3
Group 6. HFSD+NSF Rutin:Arginine 1:3
Group 7. HFSD+NSF Naringenin:Arginine:ethanol 1:3:1

Animals were kept in groups of 4 mice per cage with a 12 h/12 h light-dark cycle. Mice were identified with markings on their tails to indicate their number; these marks were renewed each time the weighing was carried out. The total study period was 100 days. During the study period, body weight and caloric intake were determined every three days by weighing the food.

All experimental groups had access to water and food ad libitum except in the fasting periods of the determinations, where only food was restricted. The water did not include any treatment.

The animals were fed with HFSD, i.e., fat (21.8%) and sucrose (21.3%), or with a control diet (AIN-93) and (sucrose 10%), for 100 days:

| Diet | Sucrose % | Fat % (added) | Kcal/g |
|---|---|---|---|
| AIN-93 | 10 | 0 | 3.9478 |
| HFSD | 21.3 | 21.8 | 4.8762 |

The NSF of flavonoids were placed directly in the food with the HFSD, according to the following table:

| | Percentage of Flavonoid | Percentage of Flavonoid |
|---|---|---|
| Flavonoid NSF | weight/weight | NSF weight/weight |
| NSF Genistein | 0.2 | 0.66 |
| NSF Fisetin | 0.2 | 0.5 |
| NSF Quercetin | 0.2 | 0.5 |
| NSF Rutin | 0.2 | 0.4 |
| NSF Naringenin | 0.2 | 0.6 |

### Weight Gain:

Food consumption and body weight of the mice were measured twice a week using an analytical scale in the case of animal weight and a grain scale in the case of food.

### Body Composition:

Mice were placed individually in a thin-walled plastic cylinder in a quantitative MRI system (Echo MRI, Houston, TX, USA). The animals were briefly subjected to a low-intensity electromagnetic field (0.05 Tesla) to measure fat mass and lean mass.

### Glucose and Insulin Levels:

Mice were subjected to a short period of fasting (7 h). Prior to glucose injection, a tail blood sample was obtained to measure serum glucose in the basal phase, after which an intraperitoneal injection of 2 g/kg body weight of glucose was performed. Tail blood samples were obtained at 15, 30, 45, 60, and 120 minutes after injection. Glucose concentration was measured with the OneTouch Ultra™ glucometer (Accu-Check Sensor, Roche Diagnostics).

### Respiratory Exchange Ratio (RER) and VO2:

Indirect calorimetry testing was carried out using a Comprehensive Laboratory Animal Monitoring System (Columbus Instruments, OH, USA). Mice were kept in individual boxes previously acclimated 24 h before the start of the study. Measurements were carried out during the 24 h after the acclimatization period. The test was carried out at 22° C. under a 12 h/12 h light-dark cycle. During the test, water was always available, and food was only available from 7:00 p.m. to 7:00 a.m. Based on the VO₂ and CO₂ measurements, energy expenditure (EE) was calculated with the following equation: EE=(3.815+1.232*RER)*VO₂.

### Biochemical Parameters:

Serum concentration of glucose, cholesterol, LDL and triglycerides were measured with a COBAS c1 11 analyzers (Roche Diagnostics, Mannheim, Germany). Serum insulin, leptin, adiponectin, and free fatty acid levels were measured using commercial kits.

Stool collection was performed 5 days before the end of the study. These collections were carried out in the light phase for 3 consecutive days. The animals were placed individually in previously sterilized container cups for individual collection of feces.

### Statistical Analysis:

Data were analyzed using the one-way ANOVA test, comparing the treated groups with the control group. Subsequently, a Fisher post hoc test was applied. The p value was taken as a significant value when it was less than 0.05.

### Results

### Weight Gain:

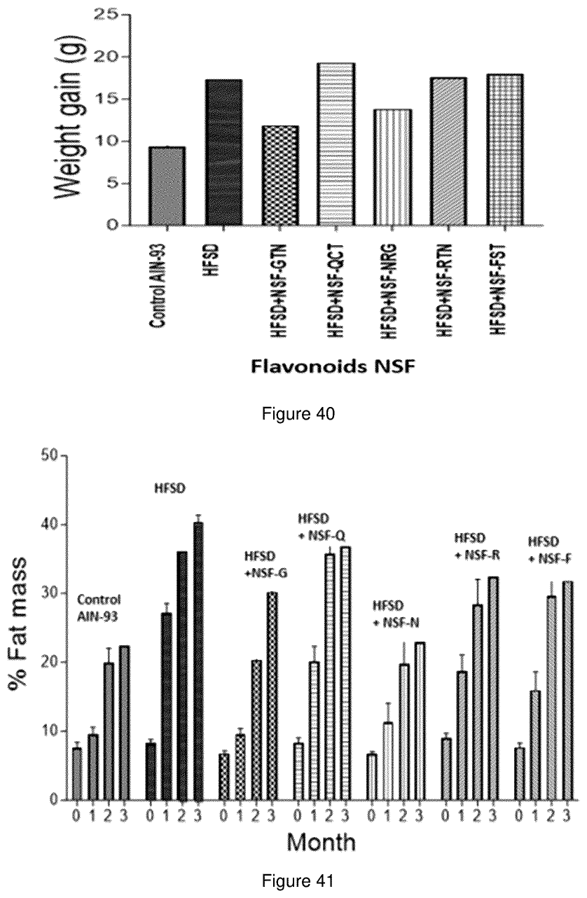

The group of mice fed with HFSD had a greater weight gain compared to animals fed normal diet (Control AIN-93). It is important to highlight that the groups fed with HFSD+NSF Naringenin (HFSD+NFS-NRG) and HFSD+NSF Genistein (HFSD+NFS-GTN) had a lower weight gain than the group fed with HFSD despite being fed with HFSD (FIG. 40).

### Body Composition:

The group fed with HFSD alone showed an increase in fat percentage from the first month, compared to the groups that received HFSD+flavonoid NSF and the control group on a standard diet (AIN-93), as shown in FIG. 41. The results show that the flavonoid NSF reduces the increase in fat percentage caused by HFSD from the first month of the study. Surprisingly, the group fed HFSD+NSF Genistein (HFSD+NSF-G) and HFSD+NSF Naringenin (HFSD+NSF-N) showed similar fat percentage values to the group that followed the standard control diet.

In the second month, a decrease in fat percentage gain was observed in all groups that received the flavonoid NSF, counteracting the effect induced by HFSD. Surprisingly, the groups supplemented with Genistein NSF and Naringenin NSF maintained comparable fat percentage values to those of the group fed with the standard control diet.

In the third month, it is observed that the groups fed with HFSD+ flavonoid NSF continue to reduce fat gain, highlighting again the group fed with HFSD+ Naringenin NSF, whose values are very similar to those of the control group fed with the standard diet (FIG. 41).

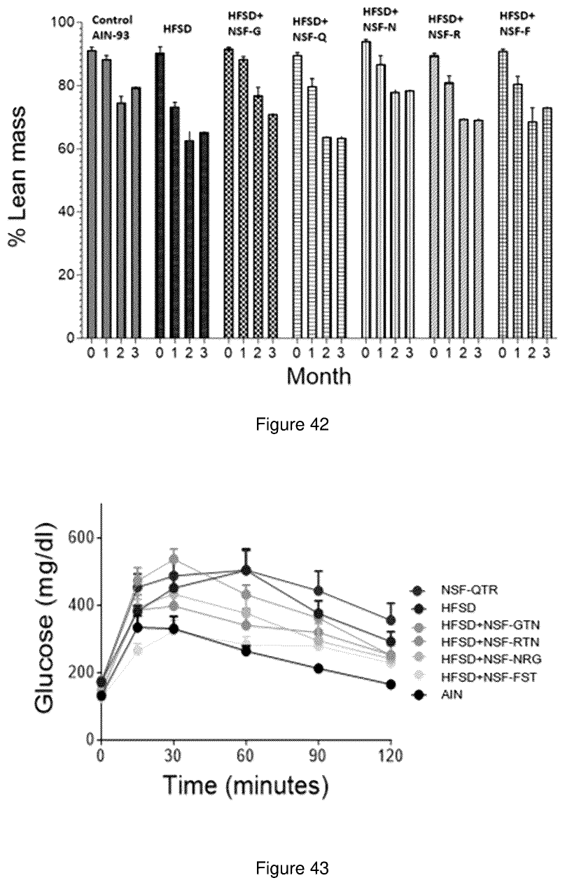

When comparing FIGS. 41 and 42 it is noted that the groups that gained more fat mass proportionally lost lean mass. Supplementation with flavonoid NSF decreased the loss of lean mass induced by HFSD from the first month.

As shown in FIG. 42, the groups fed with HFSD+Genistein NSF (HFSD+NSF-G) and HFSD+Naringenin NSF (HFSD+NSF-N) show a lower loss of lean mass, showing a similar behavior to the control group (AIN-93 diet), suggesting a beneficial effect. FIGS. 41 and 42 show that the flavonoid NSF have a beneficial effect in regulating fat gain, despite consuming a high-fat diet. The groups receiving Naringenin NSF and Genistein NSF were able to reduce both fat gain and loss of lean mass over a three-month period. These findings underscore the importance of considering the potential benefit of flavonoid NSF.

### Glucose and Insulin Levels:

In the glucose tolerance test (FIG. 43), an increase in serum glucose levels was observed after intraperitoneal administration. The highest glucose levels were recorded in the HFSD group, as well as in the groups fed with Quercetin NSF and Genistein NSF. Lower values in glucose concentrations were observed in the Fisetin NSF, Naringenin NSF and Rutin NSF groups despite having consumed HFSD.

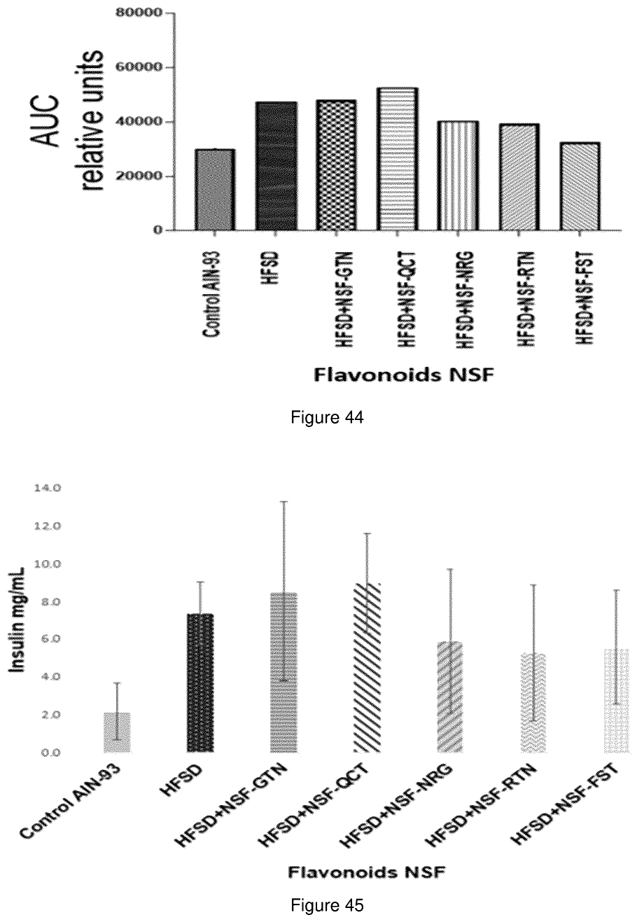

A similar trend to the glucose tolerance test is shown in the area under the curve (AUC) of FIG. 44.

In the test of serum insulin concentrations, an increase in insulin levels was observed in the groups that consumed HFSD, Quercetin NSF and Genistein NSF. The Fisetin NSF, Rutin NSF and Naringenin NSF groups show a lower insulin level compared to the HFSD. These results indicate that supplementation with the mentioned new solid forms reverses to some extent the effects of HFSD on insulin resistance (FIG. 45).

### Respiratory Exchange Ratio (RER) and VO₂:

The respiratory quotient (RER) allows us to know the type of nutrient or substrate that is being oxidized in the body as the main source of energy. When its value is close to 1, the body is metabolizing mainly carbohydrates, whereas a value close to 0.7 indicates that it is mainly metabolizing fats.

Respiratory exchange ratio (RER) in mice fed with a fasting control diet was 0.75, which corresponds to a use of energy from lipids. This coefficient increases in the postprandial period until reaching 1, which indicates that in the presence of food, the main source of energy consumption is carbohydrates. This effect is modified with the consumption of HFSD, where, despite receiving food, the mice are not able to use carbohydrates as a source of energy, which shows metabolic inflexibility. The groups fed with Fisetin NSF showed less metabolic inflexibility (FIG. 46).

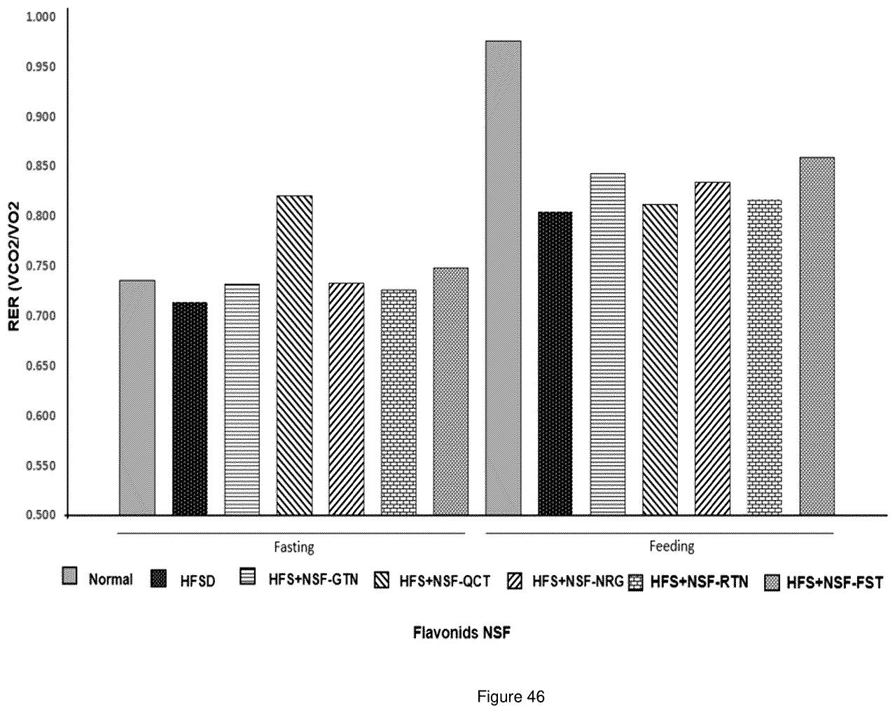

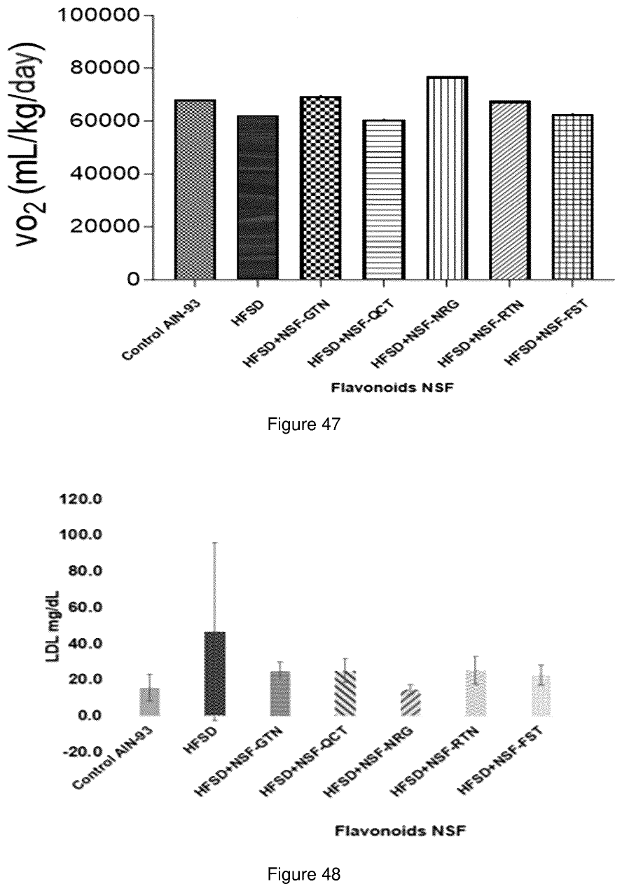

VO₂ consumption is a measurement used to determine energy expenditure through respiratory oxygen consumption. Increased VO₂ levels indicate greater energy expenditure. Oxygen consumption (VO₂) increases with Naringenin NSF and Genistein NSF compared to the control group (AIN-93). The groups fed with Rutin NSF, Fisetin NSF and Quercetin NSF show lower oxygen consumption (FIG. 47). The group fed with HFSD+Naringenin NSF had the highest VO₂ consumption, indicating greater energy expenditure.

### Response to Flavonoid NSF Chronic Consumption on LDL Concentration:

LDL concentrations were elevated only in the group that consumed the HFSD compared to the control group and the other groups that were fed with the flavonoid NSF (FIG. 48). These results show the importance of considering the potential benefit of the lipid-lowering effect of NSF flavonoid.

### Response to Flavonoid NSF Chronic Consumption on the Concentration of Liver Enzymes AST U/L:

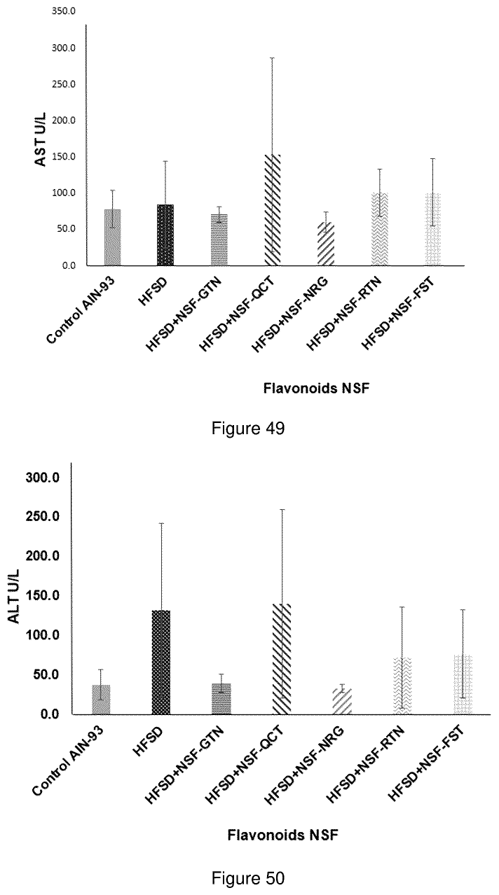

Serum concentrations of liver enzymes such as AST (aspartate amino transferase) were increased with the consumption of an HFSD. In the groups fed with Genistein NSF and Naringenin NSF, a decrease in the concentration of liver enzymes AST U/L was observed compared to the control group (AIN-93) and with Quercetin NSF (FIG. 49).

### Response of Chronic Consumption of NSF Flavonoids on the Concentration of Liver Enzymes ALT U/L:

Serum concentrations of liver enzymes such as ALT (alanine amino transferase) were increased with the consumption of an HFSD and with Quercetin NSF. In the groups fed with Genistein NSF and Naringenin NSF, a similar or lower concentration of liver enzymes ALT U/L is observed compared to the control group (FIG. 50).

### Response to Chronic Consumption of NSF Flavonoids on Urea and Creatinine Concentration:

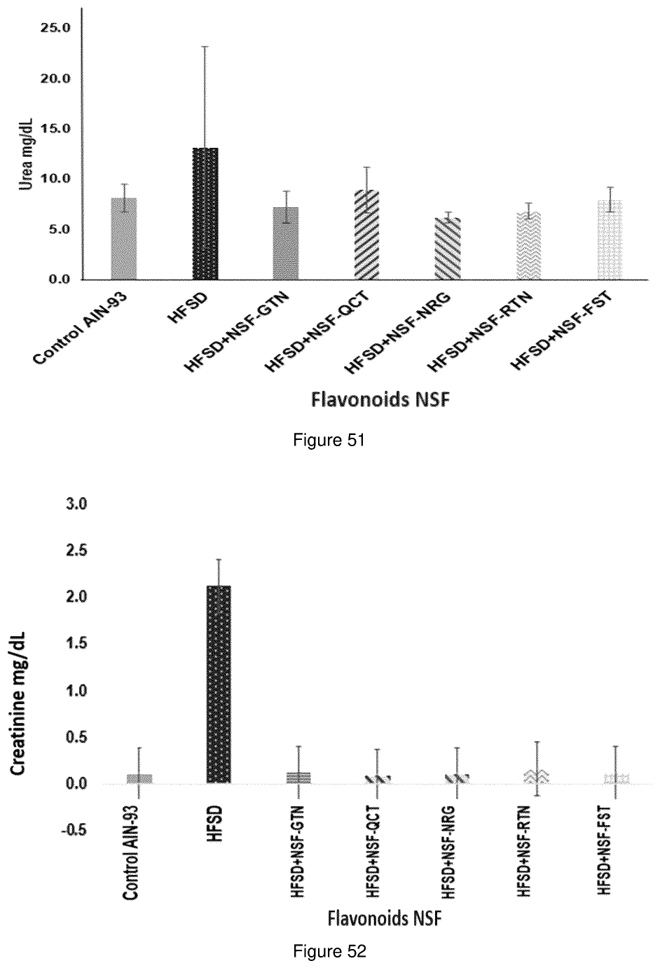

In the groups fed with the flavonoid NSF, a similar or lower concentration of urea and creatinine was observed compared to the control diet group (FIGS. 51, 52). In the groups fed with flavonoid NSF, a significant decrease in the concentration of urea and creatinine was observed compared to the HFSD group. The other groups do not present differences between them. This is relevant since all flavonoid NSF had a very pronounced positive effect on kidney function that is affected with HFSD.

### Relevance of the Results:

In the field of scientific research focused on health, the search for strategies that counteract the adverse effects of diets rich in fats and sugars is of utmost importance, given the increasing prevalence of metabolic diseases and obesity in the population. In particular, the use of natural substances such as flavonoids has emerged as a potential therapeutic tool due to their natural origin and the fact that their use has few or no side effects.

In this study, the beneficial effect of NSF supplementation of various flavonoids during HFSD was demonstrated. It was observed that all flavonoid NSF tested in this study had a beneficial effect on the body composition of the animals. Likewise, all NSF notably decreased the levels of the enzyme alanine aminotransferase (ALT), LDL levels, and urea and creatinine levels.

These results are of utmost importance as they indicate that flavonoid NSF had a hepatoprotective and nephroprotective effect during HFSD intake. Importantly, nephropathy is a serious complication of metabolic syndrome and obesity increases the risk of non-alcoholic fatty liver disease.

It was also observed that the NSF of Genistein and Naringenin reduced the weight gain induced by HFSD. In the case of the glucose tolerance test, supplementation with Fisetin NSF had an antihyperglycemic effect. The above results are complemented by plasma insulin measurements where it was observed that the NSF of Naringenin, Rutin and Fisetin decreased the levels of this hormone that are elevated in a HFSD. This reduction in insulin levels could indicate an improvement in insulin sensitivity, a key factor in metabolic syndrome. Consequently, the Flavonoid NSF examined in this study are shown as possible adjuvants to mitigate the detrimental health effects of a high-calorie diet.

The search for strategies that reduce the adverse effects of diets rich in fats and sugars is very important, given the increasing prevalence of metabolic diseases and obesity in the population. In particular, the use of natural substances such as flavonoids has emerged as a potential therapeutic tool due to their natural origin and the fact that their use has few or no side effects.

In this study, the beneficial effect of supplementation using the new solid phases of flavonoids of the present invention was demonstrated in animals fed a high-fat diet (HFSD). It was observed that all flavonoid NSF exhibit a beneficial effect on the body composition of animals. Also, Flavonoid NSF markedly decreased the levels of the enzyme alanine aminotransferase (ALT), LDL levels and urea and creatinine levels.

The results show that the flavonoid NSF had a hepatoprotective and nephroprotective effect during the intake of HFSD. Importantly, nephropathy is a serious complication of metabolic syndrome and obesity increases the risk of non-alcoholic fatty liver disease.

Consequently, the Flavonoid NSF of the present invention are shown as possible adjuvants to reduce the harmful effects on health of a high-calorie diet or diet high in fat and sugar. These NSF can be used in a composition along with pharmaceutically acceptable excipients, wherein the composition is suitable to be used as a pharmaceutical compositions, or within food products, nutritional supplements and the like.

The invention also refers to a method for improving health or nutrition in an animal or human in need thereof, the method comprising administering a composition containing at least one of the NSF of flavonoids.

The invention also refers to a method for providing antioxidant, anti-inflammatory, analgesic, antitumoral, anticancer effect in an animal or human in need thereof, the method comprising administering a composition containing at least one of the NSF of flavonoids.

The invention also refers to a method for treating metabolic syndrome in a human or animal in need thereof, comprising administering a composition containing at least one of the NSF of flavonoids.

All publications and patents referred to herein are incorporated by reference. Various modifications and variations of the described subject matter will be apparent to those skilled in the art without departing from the scope and spirit of the invention. Although the invention has been described in connection with specific embodiments, it should be understood that the invention as claimed should not be unduly limited to these embodiments. Indeed, various modifications for carrying out the invention are obvious to those skilled in the art and are intended to be within the scope of the following claims.

What is claimed is:

1.-13. (canceled)

14. A new co-amorphous solid form comprising a flavonoid and an amino acid, and solvates thereof.

15. The new co-amorphous solid form of claim 14, wherein:
the flavonoid is selected from quercetin and fisetin,
the amino acid is arginine, and
the flavonoid:arginine molar ratio is from 1:3 to 1:9,
and solvates thereof.

16. The new co-amorphous solid form of claim 14, wherein:
the flavonoid is rutin,
the amino acid is arginine, and
the flavonoid:arginine molar ratio is from 1:1 to 1:4,
and solvates thereof.

17. The new co-amorphous solid form of claim 14, wherein:
the flavonoid is genistein,
the amino acid is arginine, and
the flavonoid:arginine molar ratio is from 1:3 to 1:10,
and solvates thereof.

18. The new co-amorphous solid form of claim 14, wherein:
the flavonoid is naringenin,
the amino acid is arginine, and
the flavonoid:arginine molar ratio is 1:3,
and solvates thereof.

19. The new co-amorphous solid form of claim 14, wherein:
the flavonoid is apigenin,
the amino acid is arginine, and
the flavonoid:arginine molar ratio is selected from 1:3 and 1:8,
and solvates thereof.

20. The new co-amorphous solid form of claim 14, wherein:
the flavonoid is selected from rutin and naringenin,
the amino acid is lysine, and
flavonoid:lysine molar ratio is 1:3,
and solvates thereof.

21. A composition containing the new co-amorphous solid form of claim 14 and one or more acceptable excipients, wherein the composition is suitable to be used as a pharmaceutical composition, or within food products, nutritional supplements and the like.

22. A composition containing the new co-amorphous solid form of claim 15 and one or more acceptable excipients, wherein the composition is suitable to be used as a pharmaceutical composition, or within food products, nutritional supplements and the like.

23. A composition containing the new co-amorphous solid form of claim 16 and one or more acceptable excipients, wherein the composition is suitable to be used as a pharmaceutical composition, or within food products, nutritional supplements and the like.

24. A composition containing the new co-amorphous solid form of claim 17 and one or more acceptable excipients, wherein the composition is suitable to be used as a pharmaceutical composition, or within food products, nutritional supplements and the like.

25. A composition containing the new co-amorphous solid form of claim 18 and one or more acceptable excipients, wherein the composition is suitable to be used as a pharmaceutical composition, or within food products, nutritional supplements and the like.

26. A composition containing the new co-amorphous solid form of claim 19 and one or more acceptable excipients, wherein the composition is suitable to be used as a pharmaceutical composition, or within food products, nutritional supplements and the like.

27. A composition containing the new co-amorphous solid form of claim 20 and one or more acceptable excipients, wherein the composition is suitable to be used as a pharmaceutical composition, or within food products, nutritional supplements and the like.

28. A method for treating metabolic syndrome and/or for improving intestinal microbiota in a human or animal in need thereof, comprising administering a composition containing the new co-amorphous solid form of claim 14.

29. A method for treating metabolic syndrome and/or for improving intestinal microbiota in a human or animal in need thereof, comprising administering a composition containing the new co-amorphous solid form of claim 15.

30. A method for treating metabolic syndrome and/or for improving intestinal microbiota in a human or animal in need thereof, comprising administering a composition containing the new co-amorphous solid form of claim 16.

31. A method for treating metabolic syndrome and/or for improving intestinal microbiota in a human or animal in need thereof, comprising administering a composition containing the new co-amorphous solid form of claim 17.

32. A method for treating metabolic syndrome and/or for improving intestinal microbiota in a human or animal in need thereof, comprising administering a composition containing the new co-amorphous solid form of claim 18.

33. A method for treating metabolic syndrome and/or for improving intestinal microbiota in a human or animal in need thereof, comprising administering a composition containing the new co-amorphous solid form of claim 19.

34. A method for treating metabolic syndrome and/or for improving intestinal microbiota in a human or animal in need thereof, comprising administering a composition containing the new co-amorphous solid form of claim 20.
